(19) United States
(12) Patent Application Publication
(10) Pub. No.: US 2023/0225888 A1
(43) Pub. Date: Jul. 20, 2023

(54) BRAIDED STENT WITH IMPROVED FLEXIBILITY

(71) Applicant: BOSTON SCIENTIFIC SCIMED, INC., Maple Grove, MN (US)

(72) Inventors: Martin Murray, Loughrea (IE); Paul E. Tierney, Athenry (IE); Michael Walsh, Galway (IE); Garrett Casserly, Galway (IE); David Collins, Galway (IE); Kevin Windheuser, Hopkinton, MA (US); Molly Solomon, Groton, MA (US)

(73) Assignee: BOSTON SCIENTIFIC SCIMED, INC., Maple Grove, MN (US)

(21) Appl. No.: 18/155,996

(22) Filed: Jan. 18, 2023

Related U.S. Application Data
(60) Provisional application No. 63/300,810, filed on Jan. 19, 2022.

(51) Int. Cl. A61F 2/88

(52) U.S. Cl. CPC A61F 2/88; A61F 2230/0091

(57) ABSTRACT

A stent includes an elongated tubular member expandable from a radially collapsed configuration to a radially expanded configuration, the elongate tubular member including a first plurality of filaments extending in a first helical direction and a second plurality of filaments extending in a second helical direction, the first plurality of filaments extending in the first helical direction and the second plurality of filaments extending in the second helical direction overlapping to form a plurality of cells arranged in rows extending circumferentially about the elongated tubular member. At least some of the cells within one or more rows are adapted to provide increased flexibility to the stent.

### CROSS-REFERENCE TO RELATED APPLICATIONS

This application claims the benefit of U.S. Provisional Patent Application Serial No. 63/300,810, filed Jan. 19, 2022, which is incorporated herein by reference.

### TECHNICAL FIELD

The present disclosure pertains to medical devices, and methods for manufacturing and using medical devices. More particularly, the disclosure is directed to stents for implantation in a body lumen, and associated methods.

### BACKGROUND

Implantable medical devices (e.g., expandable stents) may be designed to treat a variety of medical conditions in the body. For example, some expandable stents may be designed to radially expand and support a body lumen and/or provide a fluid pathway for digested material, blood, or other fluid to flow therethrough following a medical procedure. Some medical devices may include radially or self-expanding stents which may be implanted transluminally via a variety of medical device delivery systems. These stents may be implanted in a variety of body lumens such as coronary or peripheral arteries, the esophageal tract, gastrointestinal tract (including the intestine, stomach and the colon), tracheobronchial tract, urinary tract, biliary tract, vascular system, etc. In some instances it may be desirable to design stents to include sufficient flexibility while maintaining sufficient radial force to open the body lumen at the treatment site.

Therefore, in some instances it may be desirable to design a stent with improved flexibility. Examples of medical devices including improved flexibility are disclosed herein.

### SUMMARY

This disclosure provides design, material, manufacturing method, and use alternatives for medical devices. As an example, a stent includes an elongated tubular member that is expandable from a radially collapsed configuration to a radially expanded configuration, the elongate tubular member including a first plurality of filaments extending in a first helical direction and a second plurality of filaments extending in a second helical direction, the first plurality of filaments extending in the first helical direction and the second plurality of filaments extending in the second helical direction overlapping to form a plurality of cells arranged in rows extending circumferentially about the elongated tubular member. At least some of the cells within one or more rows are adapted to provide increased flexibility to the stent.

Alternatively or additionally, at least some of the first plurality of filaments and at least some of the second plurality of filaments within one or more rows of the plurality of rows may be cut away to disrupt at least some of the cells, thereby increasing flexibility of the stent.

Alternatively or additionally, at least some of the plurality of cells may have a generally diamond shape having four sides formed by a pair of filaments of the first plurality of filaments extending in the first helical direction and a pair of filaments of the second plurality of filaments extending in the second helical direction.

Alternatively or additionally, a disrupted cell may include a cell that has had at least one of the four sides of the generally diamond shape removed.

Alternatively or additionally, the stent may further include a polymeric covering extending along the elongate tubular member.

Alternatively or additionally, one or more rows of cells may include at least one intact cell and a plurality of disrupted cells.

Alternatively or additionally, one or more rows of cells may include only disrupted cells, thereby separating the elongate tubular member into two or more distinct segments.

Alternatively or additionally, a first segment of the two or more distinct segments may have a first end having a plurality of cells within a first terminal row, a second segment of the two or more distinct segments may have a second end having a plurality of cells within a second terminal row, and the second segment may be rotated relative to the first segment such that the plurality of cells within the first terminal row nest between the plurality of cells within the second terminal row.

Alternatively or additionally, a first segment of the two or more distinct segments may have a first braiding pattern and a second segment of the two or more distinct segments have a second braiding pattern that is different from the first braiding pattern.

Alternatively or additionally, the first braiding pattern may differ from the second braiding pattern in one or more of filament count, braid angle, and filament diameter.

Alternatively or additionally, the two or more distinct segments may be joined together via a fixation element woven between adjacent segments.

Alternatively or additionally, the fixation element may include a filament.

Alternatively or additionally, the two or more distinct segments may be formed by disrupting all of the cells within a row of cells.

As another example, a braided stent includes an elongated tubular member that is expandable from a radially collapsed configuration to a radially expanded configuration. The elongate tubular member includes a first segment having a first plurality of filaments extending in a left to right helical direction and a second plurality of filaments extending in a right to left helical direction, the first plurality of filaments and the second plurality of filaments together forming a first plurality of cells arranged in rows extending circumferentially about the first segment. The elongate tubular member includes a second segment having a third plurality of filaments extending in the left to right helical direction and a fourth plurality of filaments extending in the right to left helical direction, the third plurality of filaments and the fourth plurality of filaments together forming a second plurality of cells arranged in rows extending circumferentially about the second segment. The first segment and the second segment are coupled together in a manner that provides the braided stent with increased flexibility.

Alternatively or additionally, the first segment and the second segment may be coupled together by having one or more of the third plurality of filaments being extensions of one or more of the first plurality of filaments and/or by having one or more of the fourth plurality of filaments being extensions of one or more of the second plurality of filaments.

Alternatively or additionally, the first segment and the second segment may be coupled together by a continuous polymeric layer that extends over at least part of the first segment and at least part of the second segment.

Alternatively or additionally, the first segment may have a first end having a plurality of cells within a first terminal row and the second segment may have a second end having a plurality of cells within a second terminal row. The first segment and the second segment may be coupled together via a fixation element woven between the plurality of cells within the first terminal row and the plurality of cells within the second terminal row.

Alternatively or additionally, the braided stent may further include a third segment having a fifth plurality of filaments extending in a left to right helical direction and a sixth plurality of filaments extending in a right to left helical direction, the first plurality of filaments and the second plurality of filaments together forming a first plurality of cells arranged in rows extending circumferentially about the first segment, wherein the second segment and the third segment may be coupled are coupled together in a manner that provides the braided stent with increased flexibility.

In another example, a braided stent includes an elongated tubular member expandable from a radially collapsed configuration to a radially expanded configuration, the elongate tubular member having a plurality of cells arranged in rows that extend circumferentially about the elongated tubular member. At least some of the plurality of cells are adapted to increase flexibility of the stent.

Alternatively or additionally, the elongated tubular member may include a first plurality of filaments extending in a first helical direction and a second plurality of filaments extending in a second helical direction, the first plurality of filaments extending in the first helical direction and the second plurality of filaments extending in the second helical direction overlapping to form a plurality of cells arranged in rows extending circumferentially about the elongated tubular member, wherein at least some of the first plurality of filaments and at least some of the second plurality of filaments within one or more rows of the plurality of rows are cut away to disrupt at least some of the cells, thereby increasing flexibility of the stent.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a side view of an illustrative stent;

FIG. 1A is an enlarged view of a portion of the illustrative stent of FIG. 1;

FIG. 2 is an enlarged view of a portion of the illustrative stent of FIG. 1;

FIG. 3 is a side view of an illustrative stent;

FIG. 4 is an enlarged view of a portion of the illustrative stent of FIG. 3;

FIG. 5 is a side view of an illustrative stent;

FIG. 6 is an enlarged view of a portion of the illustrative stent of FIG. 5;

FIG. 7 is a side view of an illustrative stent;

FIG. 8 is a side view of an illustrative stent;

FIG. 9 is a side view of an illustrative stent;

FIG. 10 is a side view of an illustrative stent;

FIG. 11 is a side view of an illustrative stent;

FIG. 12 is a side view of an illustrative stent;

FIG. 12A is an enlarged view of a portion of the illustrative stent of FIG. 12;

FIG. 13 is a side view of a portion of an illustrative stent;

FIG. 13A is a schematic cross-sectional view taken along line 13A-13A of FIG. 13;

FIG. 14 is a side view of an illustrative stent;

FIG. 14A is a schematic cross-sectional view taken along line 14A-14A of FIG. 14;

FIG. 14B is a schematic cross-sectional view taken along line 14B-14B of FIG. 14;

FIG. 15 is a side view of an illustrative stent;

FIG. 15A is an enlarged view of a portion of FIG. 15;

FIG. 16 is a side view of an illustrative stent;

FIG. 17 is a side view of an illustrative stent;

FIG. 18 is a side view of an illustrative stent;

FIG. 19 is a side view of an illustrative stent;

FIG. 19A is an enlarged view of a portion of the illustrative stent of FIG. 19;

FIG. 20 is a side view of an illustrative stent; and

FIG. 20A is an enlarged view of a portion of FIG. 20.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

In some instances, it may be desirable to provide an endoluminal implant, or stent, that can deliver luminal patency in a patient with an esophageal stricture or other medical condition. Such stents may be used in patients experiencing dysphagia, sometimes due to esophageal cancer. An esophageal stent may allow a patient to maintain nutrition via oral intake during cancer treatment or palliation periods. Some stents have a woven or knitted configuration to provide good radial strength with minimal foreshortening which may be desirable in esophageal and tracheabronchial applications as well as some post-bariatric surgery applications. While the embodiments disclosed herein are discussed with reference to esophageal stents, it is contemplated that the stents described herein may be used and sized for use in other locations such as, but not limited to: bodily tissue, bodily organs, vascular lumens, non-vascular lumens and combinations thereof, such as, but not limited to, in the coronary or peripheral vasculature, trachea, bronchi, colon, small intestine, biliary tract, urinary tract, prostate, brain, stomach and the like.

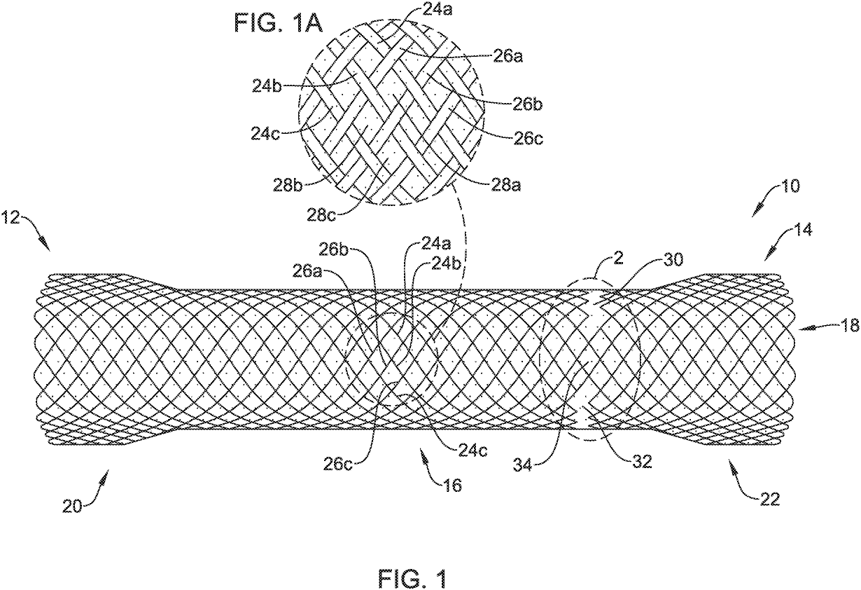

FIG. 1 is a side view of an illustrative endoluminal implant 10, such as, but not limited to, a stent. In some instances, the stent 10 may take the form of an elongated tubular member. While the stent 10 is described and shown as generally tubular, it is contemplated that the stent 10 may take any cross-sectional shape desired. The stent 10 may have a first, or proximal end 12, a second, or distal end 14 and an intermediate region 16 that is disposed between the first end 12 and the second end 14. The stent 10 may include a lumen 18 that extends form a first opening adjacent the first end 12 to a second opening adjacent the second opening 14 to allow for the passage of food, fluids, etc. to pass therethrough.

The stent 10 may be expandable from a first radially collapsed configuration (not explicitly shown) to a second radially expanded configuration. In some cases, the stent 10 may be deployed to a configuration that is between the collapsed configuration and the expanded configuration, i.e., the stent 10 may be deployed having a deployed diameter that is greater than a diameter of the stent 10 or a particular portion thereof while in its collapsed configuration yet less than a diameter of the stent 10 or a particular portion thereof while in its fully expanded configuration. In some cases, the anatomy in which the stent 10 is deployed may influence its deployed configuration. For example, if the anatomy in which the stent 10 is to be deployed has a diameter that is less than a diameter of the stent 10 or a particular portion thereof when fully expanded, the stent 10 may have a deployed diameter that is intermediate its collapsed configuration diameter and its fully expanded configuration diameter.

The stent 10 may be formed of a plurality of interwoven filaments. For example, the stent 10 may have a braided structure, fabricated from a plurality of filaments including a first plurality of filaments that each extend in a first helical direction and a second plurality of filaments that each extend in a second helical direction. It is contemplated that the filament(s) of the stent 10 may be made from a number of different materials such as, but not limited to, metals, metal alloys, shape memory alloys and/or polymers, as desired, enabling the stent 10 to be expanded into shape when accurately positioned within the anatomy. In some instances, the material may be selected to enable the stent 10 to be removed with relative ease as well. For example, the stent 10 may be formed from alloys such as, but not limited to, Nitinol and Elgiloy®. Depending on the material selected for construction of the stent 10, the stent 10 may be self-expanding, i.e., configured to automatically radially expand when unconstrained. In some instances, the stent 10 may not be self-expanding, and thus may not regain its fully expanded configuration without the assistance of an expansion device such as but not limited to an inflatable balloon disposed within the lumen 18. As used herein, the term “self-expanding” refers to the tendency of the stent 10 to return to a preprogrammed diameter when unrestrained by an external biasing force, e.g., a delivery catheter or sheath. While not shown, the stent 10 may include a one-way valve, such as an elastomeric slit valve or duck bill valve, positioned within the lumen 18 in order to prevent retrograde flow of gastrointestinal fluids, for example.

In some instances, in the radially expanded configuration, the stent 10 may include a first end region 20 proximate the first end 12 and a second end region 22 proximate the second end 14. In some cases, as illustrated, the first end region 20 and the second end region 22 may include retention features or anti-migration flared regions having enlarged diameters relative to the intermediate region 16. The anti-migration flared regions, which may be positioned adjacent to the first end 12 and/or the second end 14, may be configured to engage an interior portion of the walls of the esophagus or other body lumen. In some cases, the retention features, or flared regions, may have a larger diameter than the intermediate region 16 of the stent 10 in order to prevent the stent 10 from migrating once placed in the esophagus or other body lumen. In some instances, a transition from the cross-sectional area of the intermediate region 16 to the retention features or flared regions may be gradual, sloped, or occur in an abrupt step-wise manner, as desired.

In some instances, the first anti-migration flared region may have a first outer diameter and the second anti-migration flared region may have a second outer diameter. In some instances, the first and second outer diameters may be approximately the same, while in other instances, the first and second outer diameters may be different. In some cases, the stent 10 may include only one or none of the anti-migration flared regions. For example, the first end region 20 may include an anti-migration flare while the second end region 22 may have an outer diameter similar to the intermediate region 16. It is further contemplated that the second end region 22 may include an anti-migration flare while the first end region 20 may have an outer diameter similar to an outer diameter of the intermediate region 16. In some embodiments, the stent 10 may have a uniform outer diameter from the first end 12 to the second end 14. In some embodiments, the outer diameter of the intermediate region 16 may be in the range of 15 to 25 millimeters in the fully expanded configuration. The outer diameter of the anti-migration flares may be in the range of 20 to 30 millimeters in the fully expanded configuration. It is contemplated that the outer diameter of the stent 10 may be varied to suit the desired application.

In some cases, the stent 10 includes a first plurality of filaments that extend in a first helical direction and a second plurality of filaments that extend in a second helical direction. The stent 10 includes, for example, individual filaments 24a, 24b and 24c, each extending in a first helical direction. The stent 10 includes additional filaments (not referenced) extending in the first helical direction. The first helical direction may be considered as extending left to right, or proximal to distal, in a clockwise direction. The stent 10 includes, for example, individual filaments 26a, 26b and 26c, each extending in a second helical direction. The stent 10 includes additional filaments (not referenced) extending in the second helical direction. The second helical direction may be considered as extending right to left, or distal to proximal, in a clockwise direction.

With reference to FIG. 1A, it can be seen that the individual filaments are braided, i.e., the individual braids extend over and under each other. For example, the individual filament 24a extends under the individual filament 26a, extends over the individual filament 26b, under the individual filament 26c, and so on. Similar relationships exist for each of the individual filaments forming the stent 10. It will be appreciated that a cell 28a is formed by the intersections of the individual filaments 24a, 24b, 26a and 26b. A cell 28b is formed by the intersections of the individual filaments 24b, 24c, 26a and 26b. A cell 28c is formed by the intersections of the individual filaments 24b, 24c, 26b and 26c. Each of the cells 28a, 28b and 28c may be considered as being diamond-shaped, having four sides that are roughly equal in length. In some cases, depending on the relative angles at which the first and second helical directions extend, respectively, at least some of the cells may have two sides that are somewhat shorter and two sides that are somewhat longer than the other, for example. The cell 28a and the cell 28c may be considered as being within a single row extending circumferentially about the stent 10 while the cell 28b may be considered as being within a neighboring row extending circumferentially about the stent 10.

The stent 10 can include any number of filaments extending in the first helical direction and any number of filaments extending in the second helical direction. In some instances, the stent 10 may have an equal number of filaments extending in the first helical direction and in the second helical direction. In some cases, the stent 10 may have a relatively greater number of filaments extending in the first helical direction and a relatively lesser number of filaments extending in the second helical direction. The stent 10 may, for example, have a relatively lesser number of filaments extending in the first helical direction and a relatively greater number of filaments extending in the second helical direction. The stent 10 may also include one or more filaments that extend in a longitudinal direction, for example. In some cases, the stent 10 may have six, seven, eight, nine, ten, eleven, twelve or more filaments extending in the first helical direction and may have six, seven, eight, nine, ten, eleven or twelve or more filaments extending in the second helical direction.

It will be appreciated that in many cases, the performance demands on a stent, such as the stent 10, can be conflicting. For example, strength, including axial strength and radial strength, versus flexibility is a common conflict in designing medical devices such as stents. Constructing a stent that can fit into a possibly tortuous anatomy can conflict with a desire to provide desired strength. Constructing a stent that will remain in place, and not migrate, can conflict with a desire to possibly be able to move or even remove a stent. These are just examples. In some cases, the stent 10 may include one or more features that can improve the flexibility of the stent 10 while retaining desired axial strength and/or radial strength.

In some cases, the flexibility of the stent 10, or at least a portion thereof, may be increased by cutting or otherwise removing sections of some of the individual filaments. For example, the stent 10 includes a first void 30 and a second void 32. In some cases, the first void 30 and the second void 32 may be distinct voids. As shown, there is a single intact cell 34 that is disposed between the first void 30 and the second void 32. There may be a similar intact cell (not visible) on the back side of the stent 10 disposed between the first void 30 and the second void 32. In some cases, the first void 30 and the second void 32 may actually be common to each other on the back side of the stent 10, with no intervening intact cell. While the first void 30 and the second void 32 align with a single circumferential row of the stent 10, in some cases the first void 30 and/or the second void 32 may be multiple rows wide. As will be discussed, in some cases the stent 10 may include multiple voids that extend at least partially circumferentially around the stent 10 and voids may be located at multiple axial positions (multiple spaced-apart rows) longitudinally separated along the stent 10.

The first void 30 and the second void 32 may extend circumferentially around the stent 10 at a first axial position of the stent 10, or each of the first void 30 and the second void 32 may extend circumferentially around the stent 10 at first and second axially spaced apart positions of the stent 10, if desired. The first void 30 and/or the second void 32 may extend circumferentially around any desired arc length of the circumference of the stent 10. For example, each of the first void 30 and/or the second void 32 may extend 30 degrees or more, 40 degrees or more, 45 degrees or more, 60 degrees or more, 75 degrees or more, 85 degrees or more, 90 degrees or more, 120 degrees or more, 150 degrees or more, or 180 degrees or more around the circumference of the stent 10.

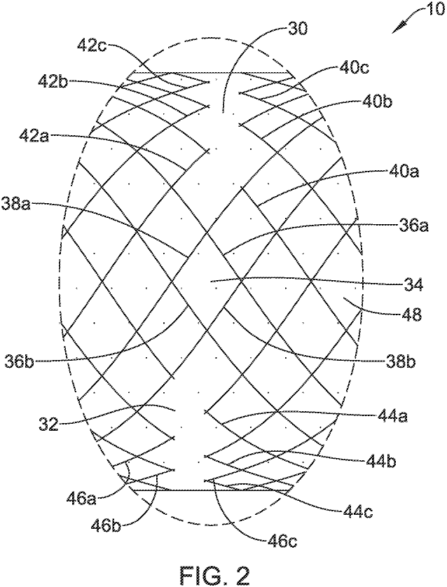

FIG. 2 provides an enlarged view of a portion of the stent 10. It can be seen that the single intact cell 34 is formed by the intersections of filaments 36a and 36b extending in the first helical direction and filaments 38a and 38b extending in the second helical direction. The first void 30 is formed by cutting or otherwise removing a portion of filaments 40a, 40b and 40c extending in the first helical direction and cutting or otherwise removing a portion of filaments 42a, 42b and 42c extending in the second helical direction. Depending on how many filaments the stent 10 includes, and the overall dimensions of the first void 30, additional filaments extending in the first helical direction and/or in the second helical direction may also have portions thereof cut away or otherwise removed. The second void 32 is formed by cutting or otherwise removing a portion of filaments 44a, 44b and 44c extending in the first helical direction and cutting or otherwise removing a portion of filaments 46a, 46b and 46c extending in the second helical direction. The cells that are no longer intact ay be considered as being disrupted cells, i.e., cells previously defined by a removed portion of a filament of the braided tubular member, thereby merging a plurality of cells together with no intervening filament therebetween.

The disrupted cells may be formed by cutting away portions of the individual filaments, and thus merging a plurality of cells together with no intervening filament therebetween. This may include laser cutting the individual filaments. While not expressly shown, in some cases at least some of the cut filament ends may be welded together to secure the remaining cells. In some cases, this may include saw cutting or even grinding the individual filaments. In some instances, regardless of the procedure used in removing portions of the individual filaments, the individual filaments may have cut ends that extend slightly beyond an intersecting filament. As an example, looking at the missing section cut out of the individual filament 40b, it can be seen that the cut ends of the individual filament 40b extend slightly beyond the individual filaments 42a and 42b.

The disrupted cells may be formed at any stage of manufacturing the stent 10. In some cases, the stent 10 is braided and annealed to set the remembered shape of the stent 10 prior to forming any of the disrupted cells. By annealing the stent 10 prior to cutting away any portions of any of the individual filaments, the cut ends of any cut filaments will tend to “remember” their shape, and thus remain in place. In some instances, an inner surface and/or an outer surface of the stent 10 may be entirely, substantially, or partially covered with a polymeric covering or coating 48 (shown via a dotted pattern). The covering or coating 48 may span both the intact cells as well as the disrupted cells. The covering or coating 48 may help reduce food impaction and/or tumor or tissue ingrowth, for example. In some cases, the covering or coating 48 may be dip coated onto the stent 10 after the voids 30 and 32 have been formed. The covering or coating 48 may be spray coated onto the stent 10 after the voids 30 and 32 have been formed. In some cases, the covering or coating 48 may be formed of any desired polymeric material. The covering or coating 48 may also help the stent 10 to retain its shape even after filaments have been cut away or otherwise removed. The covering or coating 48 may also help to prevent tissue ingrowth into the first void 30 and/or the second void 32.

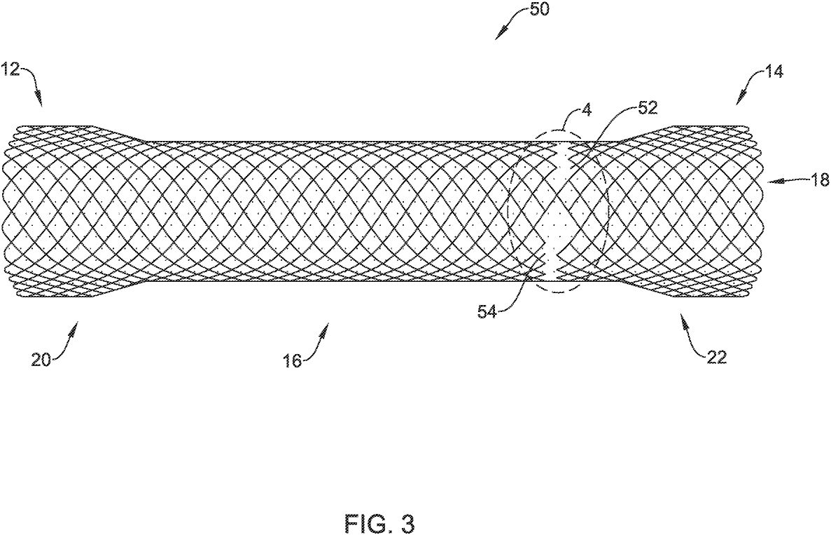

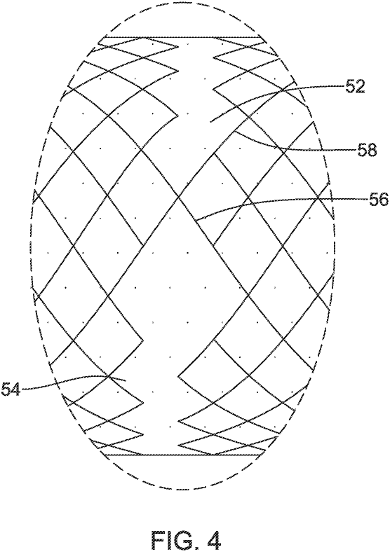

FIG. 3 is a side view of an illustrative endoluminal implant 50, such as but not limited to, a stent. The stent 50 may take the form of an elongated tubular member, although the stent 50 may take any cross-sectional shape desired. For example, the stent 50 may have a braided structure, fabricated from a plurality of filaments including a first plurality of filaments that each extend in a first helical direction and a second plurality of filaments that each extend in a second helical direction.

The stent 50 may have a first, or proximal end 12, a second, or distal end 14 and an intermediate region 16 that is disposed between the first end 12 and the second end 14. The stent 50 may include a lumen 18 that extends form a first opening adjacent the first end 12 to a second opening adjacent the second end 14 to allow for the passage of food, fluids, etc. to pass therethrough.

The stent 50 may be expandable from a first radially collapsed configuration (not explicitly shown) to a second radially expanded configuration. In some cases, the stent 50 may be deployed to a configuration that is between the collapsed configuration and the expanded configuration, i.e., the stent 50 may be deployed having a deployed diameter that is greater than a diameter of the stent 50 or a particular portion thereof while in its collapsed configuration yet less than a diameter of the stent 50 or a particular portion thereof while in its fully expanded configuration. In some cases, the anatomy in which the stent 50 is deployed may influence its deployed configuration. For example, if the anatomy in which the stent 50 is to be deployed has a diameter that is less than a diameter of the stent 50 or a particular portion thereof when fully expanded, the stent 50 may have a deployed diameter that is intermediate its collapsed configuration diameter and its fully expanded configuration diameter.

In some instances, in the radially expanded configuration, the stent 50 may include a first end region 20 proximate the first end 12 and a second end region 22 proximate the second end 14. In some cases, as illustrated, the first end region 20 and the second end region 22 may include retention features or anti-migration flared regions having enlarged diameters relative to the intermediate region 16. The anti-migration flared regions, which may be positioned adjacent to the first end 12 and/or the second end 14, may be configured to engage an interior portion of the walls of the esophagus or other body lumen. In some cases, the retention features, or flared regions, may have a larger diameter than the intermediate region 16 of the stent 50 in order to prevent the stent 50 from migrating once placed in the esophagus or other body lumen. In some instances, a transition from the cross-sectional area of the intermediate region 16 to the retention features or flared regions may be gradual, sloped, or occur in an abrupt step-wise manner, as desired.

In some instances, the first anti-migration flared region may have a first outer diameter and the second anti-migration flared region may have a second outer diameter. In some instances, the first and second outer diameters may be approximately the same, while in other instances, the first and second outer diameters may be different. In some cases, the stent 50 may include only one or none of the anti-migration flared regions. For example, the first end region 20 may include an anti-migration flare while the second end region 22 may have an outer diameter similar to the intermediate region 16. It is further contemplated that the second end region 22 may include an anti-migration flare while the first end region 20 may have an outer diameter similar to an outer diameter of the intermediate region 16. In some embodiments, the stent 50 may have a uniform outer diameter from the first end 12 to the second end 14. In some embodiments, the outer diameter of the intermediate region 16 may be in the range of 15 to 25 millimeters in the fully expanded configuration. The outer diameter of the anti-migration flares may be in the range of 20 to 30 millimeters in the fully expanded configuration. It is contemplated that the outer diameter of the stent 50 may be varied to suit the desired application.

In some cases, the flexibility of the stent 50, or at least a portion thereof, may be increased by cutting or otherwise removing sections of some of the individual filaments. For example, the stent 50 includes a first void 52 and a second void 54. In some cases, the first void 52 and the second void 54 may be distinct voids. In some cases, the first void 52 and the second void 54 may actually join together on the back side of the stent 50 (not shown). As will be discussed, in some cases the stent 50 may include multiple voids that extend at least partially circumferentially around the stent 50 and voids may be located at multiple axial positions (multiple spaced-apart rows) longitudinally separated along the stent 50.

The first void 52 and the second void 54 may extend circumferentially around the stent 50 at a first axial position of the stent 50, or each of the first void 52 and the second void 54 may extend circumferentially around the stent 50 at first and second axially spaced apart positions of the stent 50, if desired. The first void 52 and/or the second void 54 may extend circumferentially around any desired arc length of the circumference of the stent 50. For example, each of the first void 52 and/or the second void 54 may extend 30 degrees or more, 40 degrees or more, 45 degrees or more, 60 degrees or more, 75 degrees or more, 85 degrees or more, 90 degrees or more, 120 degrees or more, 150 degrees or more, or 180 degrees or more around the circumference of the stent 50.

FIG. 4 provides an enlarged view of a portion of the stent 50. In contrast to the stent 10, which included a single intact cell that was formed by the intersections of filaments 36a and 36b extending in the first helical direction and the filaments 38a and 38b extending in the second helical direction (FIG. 2), the stent 50 does not include any intact cells within a circumferential row of cells including the first void 52 and the second void 54. Rather, only a single filament 56 extending in the first helical direction and a single filament 58 extending in the second helical direction are all that separate the first void 52 from the second void 54, at least on the visible portion of the stent 50. It will be appreciated that the stent 50 may have additional flexibility, at least in the region of the first void 52 and the second void 54, relative to the voids 30 and 32 shown in FIGS. 3 and 4, for example.

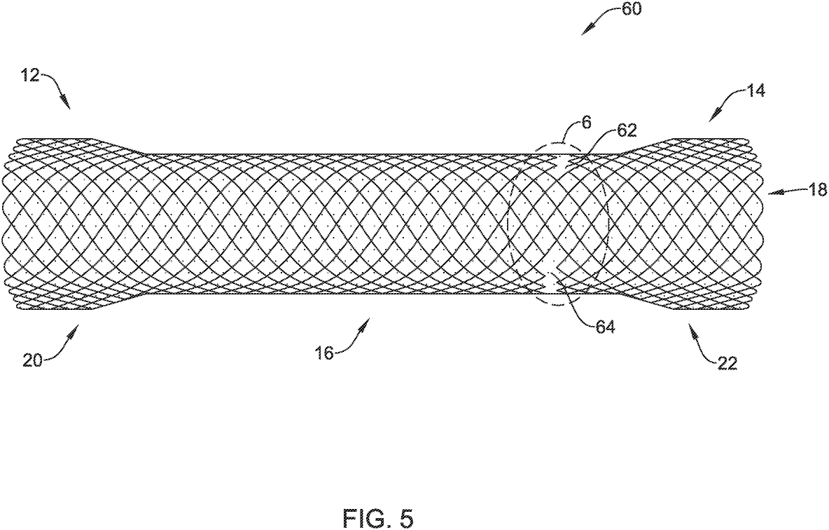

FIG. 5 is a side view of an illustrative endoluminal implant 60, such as but not limited to, a stent. The stent 60 may take the form of an elongated tubular member, although the stent 60 may take any cross-sectional shape desired. For example, the stent 60 may have a braided structure, fabricated from a plurality of filaments including a first plurality of filaments that each extend in a first helical direction and a second plurality of filaments that each extend in a second helical direction.

The stent 60 may have a first, or proximal end 12, a second, or distal end 14 and an intermediate region 16 that is disposed between the first end 12 and the second end 14. The stent 60 may include a lumen 18 that extends form a first opening adjacent the first end 12 to a second opening adjacent the second opening 14 to allow for the passage of food, fluids, etc. to pass therethrough.

The stent 60 may be expandable from a first radially collapsed configuration (not explicitly shown) to a second radially expanded configuration. In some cases, the stent 60 may be deployed to a configuration that is between the collapsed configuration and the expanded configuration, i.e., the stent 60 may be deployed having a deployed diameter that is greater than a diameter of the stent 60 or a particular portion thereof while in its collapsed configuration yet less than a diameter of the stent 60 or a particular portion thereof while in its fully expanded configuration. In some cases, the anatomy in which the stent 60 is deployed may influence its deployed configuration. For example, if the anatomy in which the stent 60 is to be deployed has a diameter that is less than a diameter of the stent 60 or a particular portion thereof when fully expanded, the stent 60 may have a deployed diameter that is intermediate its collapsed configuration diameter and its fully expanded configuration diameter.

In some instances, in the radially expanded configuration, the stent 60 may include a first end region 20 proximate the first end 12 and a second end region 22 proximate the second end 14. In some cases, as illustrated, the first end region 20 and the second end region 22 may include retention features or anti-migration flared regions having enlarged diameters relative to the intermediate region 16. The anti-migration flared regions, which may be positioned adjacent to the first end 12 and/or the second end 14, may be configured to engage an interior portion of the walls of the esophagus or other body lumen. In some cases, the retention features, or flared regions, may have a larger diameter than the intermediate region 16 of the stent 60 in order to prevent the stent 60 from migrating once placed in the esophagus or other body lumen. In some instances, a transition from the cross-sectional area of the intermediate region 16 to the retention features or flared regions may be gradual, sloped, or occur in an abrupt step-wise manner, as desired.

In some instances, the first anti-migration flared region may have a first outer diameter and the second anti-migration flared region may have a second outer diameter. In some instances, the first and second outer diameters may be approximately the same, while in other instances, the first and second outer diameters may be different. In some cases, the stent 60 may include only one or none of the anti-migration flared regions. For example, the first end region 20 may include an anti-migration flare while the second end region 22 may have an outer diameter similar to the intermediate region 16. It is further contemplated that the second end region 22 may include an anti-migration flare while the first end region 20 may have an outer diameter similar to an outer diameter of the intermediate region 16. In some embodiments, the stent 60 may have a uniform outer diameter from the first end 12 to the second end 14. In some embodiments, the outer diameter of the intermediate region 16 may be in the range of 15 to 25 millimeters in the fully expanded configuration. The outer diameter of the anti-migration flares may be in the range of 20 to 30 millimeters in the fully expanded configuration. It is contemplated that the outer diameter of the stent 60 may be varied to suit the desired application.

In some cases, the flexibility of the stent 60, or at least a portion thereof, may be increased by cutting or otherwise removing sections of some of the individual filaments. For example, the stent 60 includes a first void 62 and a second void 64. In some cases, the first void 62 and the second void 64 may be distinct voids. In some cases, the first void 62 and the second void 64 may actually join together on the back side of the stent 60 (not shown). As will be discussed, in some cases the stent 60 may include multiple voids that extend at least partially circumferentially around the stent 60 and voids may be located at multiple axial positions (multiple spaced-apart rows) longitudinally separated along the stent 60.

The first void 62 and the second void 64 may extend circumferentially around the stent 60 at a first axial position of the stent 60, or each of the first void 62 and the second void 64 may extend circumferentially around the stent 60 at first and second axially spaced apart positions of the stent 60, if desired. The first void 62 and/or the second void 64 may extend circumferentially around any desired arc length of the circumference of the stent 60. For example, each of the first void 62 and/or the second void 64 may extend 30 degrees or more, 40 degrees or more, 45 degrees or more, 60 degrees or more, 75 degrees or more, 85 degrees or more, 90 degrees or more, 120 degrees or more, 150 degrees or more, or 180 degrees or more around the circumference of the stent 60.

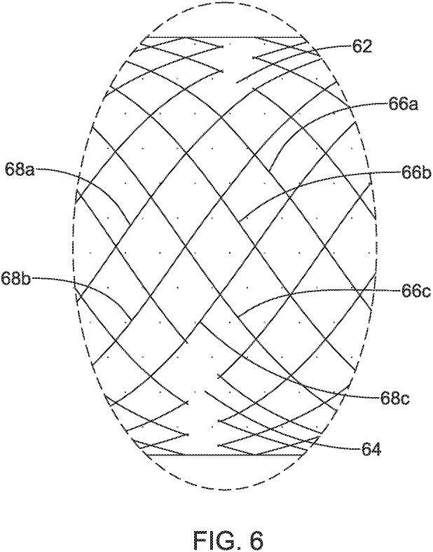

FIG. 6 provides an enlarged view of a portion of the stent 60. As shown, the stent 60 includes a total of three filaments 66a, 66b and 66c extending in the first helical direction that cross a circumferential row of cells that includes the first void 52 and the second void 54 and a total of three filaments 68a, 68b and 68c extending in the second helical direction that cross that same circumferential row of cells. Relative to the stent 10 or the stent 50, the stent 60 may exhibit slightly less flexibility but perhaps additional strength.

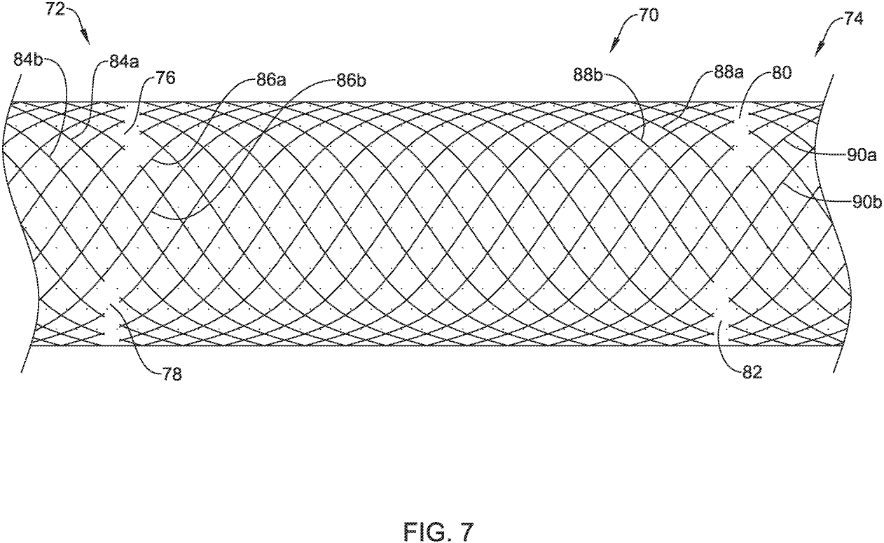

FIG. 7 is a side view of a portion of an illustrative stent 70 that includes a first or proximal region 72 and a second or distal region 74. The stent 70 may be expandable from a first radially collapsed configuration (not explicitly shown) to a second radially expanded configuration. In some cases, the stent 70 may be deployed to a configuration that is between the collapsed configuration and the expanded configuration, i.e., the stent 70 may be deployed having a deployed diameter that is greater than a diameter of the stent 70 or a particular portion thereof while in its collapsed configuration yet less than a diameter of the stent 70 or a particular portion thereof while in its fully expanded configuration. In some cases, the anatomy in which the stent 70 is deployed may influence its deployed configuration. For example, if the anatomy in which the stent 70 is to be deployed has a diameter that is less than a diameter of the stent 70 or a particular portion thereof when fully expanded, the stent 70 may have a deployed diameter that is intermediate its collapsed configuration diameter and its fully expanded configuration diameter.

In some cases, in the radially expanded configuration, the stent 70 may include anti-migration flared regions having enlarged diameters relative to a diameter of the illustrated portion of the stent 70. The anti-migration flared regions, if present, may be configured to engage an interior portion of the walls of the esophagus or other body lumen. The enlarged-diameter anti-migration regions can help to prevent the stent 70 from migrating once placed in the esophagus or other body lumen. In some instances, a transition to the enlarged diameters may be gradual, sloped, or occur in an abrupt step-wise manner, as desired.

In some instances, the first anti-migration flared region may have a first outer diameter and the second anti-migration flared region may have a second outer diameter. In some instances, the first and second outer diameters may be approximately the same, while in other instances, the first and second outer diameters may be different. In some cases, the stent 70 may include only one or none of the anti-migration flared regions. For example, a first end of the stent 70 may include an anti-migration flare while a second end of the stent 70 may not. In some cases, the second end of the stent 70 may include an anti-migration flare while the first end of the stent 70 does not. The stent 70 may have an outer diameter, outside of any flared regions, that is in the range of 15 to 25 millimeters in the fully expanded configuration. The outer diameter of any anti-migration flares may be in the range of 20 to 30 millimeters in the fully expanded configuration. It is contemplated that the outer diameter of the stent 70 may be varied to suit the desired application.

In order to enhance the flexibility of the stent 70, the stent 70 includes a first void 76 located within the proximal region 72 and a second void 78 that is located within the proximal region 72. The first void 76 and the second void 78 may each extend only a short distance circumferentially around the stent 70. For example, each of the first void 76 and/or the second void 78 may extend between 10 degrees to 60 degrees, or between 15 degrees to 50 degrees around the circumference of the stent 70. However, in other instances, the first void 76 and/or the second void 78 may extend greater than 60 degrees around the circumference of the stent 70 or less than 10 degrees around the circumference of the stent 70. In some cases, the first void 76 and the second void 78 extend all the way around the stent 70, and join together on the back side (not shown) of the stent 70. The stent 70 includes a third void 80 that is located within the distal region 74 and a fourth void 82 that is located within the distal region 74. The third void 80 and the fourth void 82 may each extend only a short distance circumferentially around the stent 70. For example, each of the third void 80 and/or the fourth void 82 may extend between 10 degrees to 60 degrees, or between 15 degrees to 50 degrees around the circumference of the stent 70. However, in other instances, the third void 80 and/or the fourth void 82 may extend greater than 60 degrees around the circumference of the stent or less than 10 degrees around the circumference of the stent 70. In some cases, the third void 80 and the fourth void 82 extend all the way around the stent 70, and join together on the back side (not shown) of the stent 70.

The relative size of the first void 76, the second void 78, the third void 80 and the fourth void 82 may be varied, depending on the intended application for the stent 70. As shown, there are a total of two filaments 84a and 84b that extend in the first helical direction and that cross a circumferential row of cells that includes the first void 76 and the second void 78, much like what is shown in FIG. 4. In some cases, there may be three such filaments extending in the first helical direction and crossing that circumferential row of cells, much like what is shown in FIG. 6. Alternatively, in some cases there may be only a single filament extending in the first helical direction and crossing that row circumferential row of cells. Changing how many filaments remain after forming the voids 76 and 78 can impact the performance of the proximal region 72 of the stent 70, for example.

Similarly, as shown, there are a total of two filaments 88a and 84b that extend in the first helical direction and that cross a circumferential row of cells that includes the third void 80 and the fourth void 82, much like what is shown in FIG. 2. In some cases, there may be three such filaments extending in the first helical direction and crossing that circumferential row of cells, much like what is shown in FIG. 6. Alternatively, in some cases there may be only a single filament extending in the first helical direction and crossing that circumferential row of cells, much like what is shown in FIG. 4. Changing how many filaments remain after forming the voids 80 and 82 can impact the performance of the distal region 74 of the stent 70, for example.

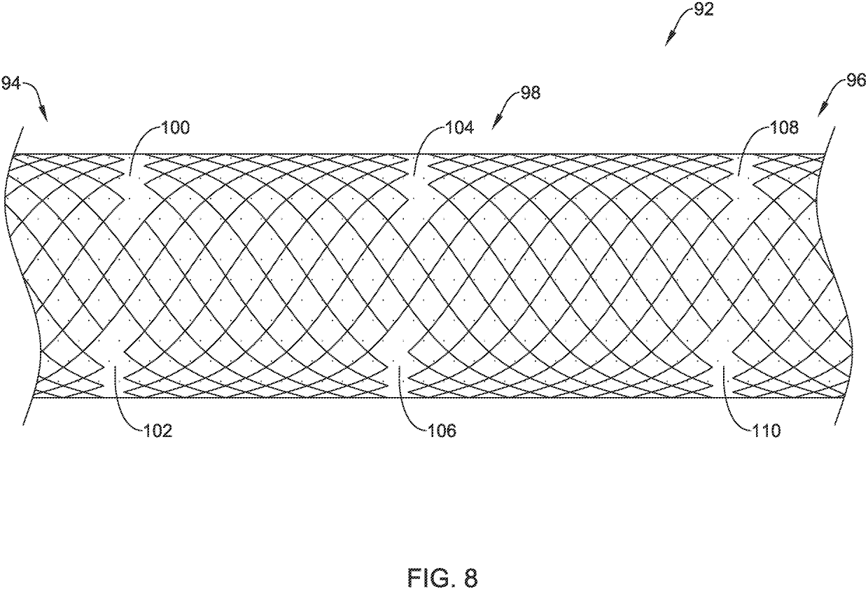

FIG. 8 is a side view of a portion of an illustrative stent 92 that includes a first or proximal region 94, a second or distal region 96 and an intervening intermediate region 98. The stent 92 may be expandable from a first radially collapsed configuration (not explicitly shown) to a second radially expanded configuration. In some cases, the stent 92 may be deployed to a configuration that is between the collapsed configuration and the expanded configuration, i.e., the stent 92 may be deployed having a deployed diameter that is greater than a diameter of the stent 92 or a particular portion thereof while in its collapsed configuration yet less than a diameter of the stent 92 or a particular portion thereof while in its fully expanded configuration. In some cases, the anatomy in which the stent 92 is deployed may influence its deployed configuration. For example, if the anatomy in which the stent 92 is to be deployed has a diameter that is less than a diameter of the stent 92 or a particular portion thereof when fully expanded, the stent 92 may have a deployed diameter that is intermediate its collapsed configuration diameter and its fully expanded configuration diameter.

In some cases, in the radially expanded configuration, the stent 92 may include anti-migration flared regions having enlarged diameters relative to a diameter of the illustrated portion of the stent 92. The anti-migration flared regions, if present, may be configured to engage an interior portion of the walls of the esophagus or other body lumen. The enlarged-diameter anti-migration regions can help to prevent the stent 92 from migrating once placed in the esophagus or other body lumen. In some instances, a transition to the enlarged diameters may be gradual, sloped, or occur in an abrupt step-wise manner, as desired.

In some instances, the first anti-migration flared region may have a first outer diameter and the second anti-migration flared region may have a second outer diameter. In some instances, the first and second outer diameters may be approximately the same, while in other instances, the first and second outer diameters may be different. In some cases, the stent 92 may include only one or none of the anti-migration flared regions. For example, a first end of the stent 92 may include an anti-migration flare while a second end of the stent 92 may not. In some cases, the second end of the stent 92 may include an anti-migration flare while the first end of the stent 92 does not. The stent 92 may have an outer diameter, outside of any flared regions, that is in the range of 15 to 25 millimeters in the fully expanded configuration. The outer diameter of any anti-migration flares may be in the range of 20 to 30 millimeters in the fully expanded configuration. It is contemplated that the outer diameter of the stent 92 may be varied to suit the desired application.

In order to enhance the flexibility of the stent 92, the stent 92 includes a first void 100 located within the proximal region 94 and a second void 102 that is located within the proximal region 94. The first void 100 and the second void 102 may each extend only a short distance circumferentially around the stent 92. For example, each of the first void 100 and/or the second void 102 may extend between 10 degrees to 60 degrees, or between 15 degrees to 50 degrees around the circumference of the stent 92. However, in other instances, the first void 100 and/or the second void 102 may extend greater than 60 degrees around the circumference of the stent 92 or less than 10 degrees around the circumference of the stent 92. In some cases, the first void 100 and the second void 102 extend all the way around the stent 92, and join together on the back side (not shown) of the stent 92. The stent 92 includes a third void 104 that is located within the intermediate region 98 and a fourth void 106 that is located within the intermediate region 98. The third void 104 and the fourth void 106 may each extend only a short distance circumferentially around the stent 92. For example, each of the third void 104 and/or the fourth void 106 may extend between 10 degrees to 60 degrees, or between 15 degrees to 50 degrees around the circumference of the stent 92. However, in other instances, the third void 104 and/or the fourth void 106 may extend greater than 60 degrees around the circumference of the stent 92 or less than 10 degrees around the circumference of the stent 92. In some cases, the third void 104 and the fourth void 106 extend all the way around the stent 92, and join together on the back side (not shown) of the stent 92. The stent 92 includes a fifth void 108 that is located within the distal region 96 and a sixth void 110 that is located within the distal region 96. The fifth void 108 and the sixth void 110 may each extend only a short distance circumferentially around the stent 92. For example, each of the fifth void 108 and/or the sixth void 110 may extend between 10 degrees to 60 degrees, or between 15 degrees to 50 degrees around the circumference of the stent 92. However, in other instances, the fifth void 108 and/or the sixth void 110 may extend greater than 60 degrees around the circumference of the stent 92, or less than 10 degrees around the circumference of the stent 92. In some cases, the fifth void 108 and the sixth void 110 extend all the way around the stent 92, and join together on the back side (not shown) of the stent 92.

Similar to what is shown in FIG. 7, there are a total of two filaments extending in the first helical direction and two filaments extending in the second helical direction that cross each of the circumferential rows of cells in which the first void 100 and the second void 102, the third void 104 and the fourth void 106, and the fifth void 108 and the sixth void 110, respectively, are located. This is similar to what is shown in FIG. 2. In some cases, there may be three such filaments extending in the first helical direction and crossing each circumferential row of cells that includes voids, much like what is shown in FIG. 6. Alternatively, in some cases there may be only a single filament extending in the first helical direction and crossing that circumferential row of cells, similar to what is shown in FIG. 4.

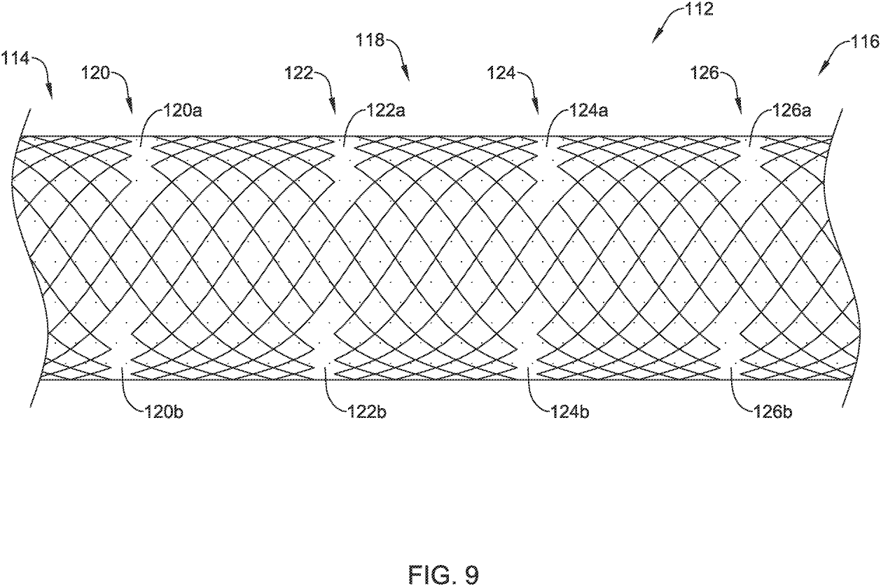

FIG. 9 is a side view of a portion of an illustrative stent 112 that includes a first or proximal region 114, a second or distal region 116 and an intervening intermediate region 118. The stent 112 may be expandable from a first radially collapsed configuration (not explicitly shown) to a second radially expanded configuration. In some cases, the stent 112 may be deployed to a configuration that is between the collapsed configuration and the expanded configuration, i.e., the stent 112 may be deployed having a deployed diameter that is greater than a diameter of the stent 112 or a particular portion thereof while in its collapsed configuration yet less than a diameter of the stent 112 or a particular portion thereof while in its fully expanded configuration. In some cases, the anatomy in which the stent 112 is deployed may influence its deployed configuration. For example, if the anatomy in which the stent 112 is to be deployed has a diameter that is less than a diameter of the stent 112 or a particular portion thereof when fully expanded, the stent 112 may have a deployed diameter that is intermediate its collapsed configuration diameter and its fully expanded configuration diameter.

In some cases, in the radially expanded configuration, the stent 112 may include anti-migration flared regions having enlarged diameters relative to a diameter of the illustrated portion of the stent 112. The anti-migration flared regions, if present, may be configured to engage an interior portion of the walls of the esophagus or other body lumen. The enlarged-diameter anti-migration regions can help to prevent the stent 112 from migrating once placed in the esophagus or other body lumen. In some instances, a transition to the enlarged diameters may be gradual, sloped, or occur in an abrupt step-wise manner, as desired.

In some instances, the first anti-migration flared region may have a first outer diameter and the second anti-migration flared region may have a second outer diameter. In some instances, the first and second outer diameters may be approximately the same, while in other instances, the first and second outer diameters may be different. In some cases, the stent 112 may include only one or none of the anti-migration flared regions. For example, a first end of the stent 112 may include an anti-migration flare while a second end of the stent 112 may not. In some cases, the second end of the stent 112 may include an anti-migration flare while the first end of the stent 112 does not. The stent 112 may have an outer diameter, outside of any flared regions, that is in the range of 15 to 25 millimeters in the fully expanded configuration. The outer diameter of any anti-migration flares may be in the range of 20 to 30 millimeters in the fully expanded configuration. It is contemplated that the outer diameter of the stent 112 may be varied to suit the desired application.

In order to enhance the flexibility of the stent 112, the stent 112 includes a plurality of flexibility-enhancing rows of cells in which at least some of the cells within each of the flexibility-enhancing rows have been disrupted. The stent 112 includes a first flexibility-enhancing row 120 that is located within the proximal region 114 of the stent 112, a second flexibility-enhancing row 122 that is located within the intermediate region 118, a third flexibility-enhancing row 124 that is located within the intermediate region 118, and a fourth flexibility-enhancing row 126 that is located within the distal region 116. While a total of four flexibility-enhancing rows 120, 122, 124, 126 are illustrated, it will be appreciated that this is merely illustrative, as the stent 112 may have any desired number of flexibility-enhancing rows, including five, six, seven, eight or more flexibility-enhancing rows. In some instances, the stent 112 may have fewer than four flexibility-enhancing rows.

As seen, the first flexibility-enhancing row 120 includes a void 120a and a void 120b. The voids 120a and 120b may each extend only a short distance circumferentially around the stent 112. In some cases, the voids 120a and 120b may extend all the way around the stent 112 and join together on the back side (not shown) of the stent 112. The second flexibility-enhancing row 122 includes a void 122a and a void 122b. The voids 122a and 122b may each extend only a short distance circumferentially around the stent 112. In some cases, the voids 122a and 122b may extend all the way around the stent 112 and join together on the back side (not shown) of the stent 112. The third flexibility-enhancing row 124 includes a void 124a and a void 124b. The voids 124a and 124b may each extend only a short distance circumferentially around the stent 112. In some cases, the voids 124a and 124b may extend all the way around the stent 112 and join together on the back side (not shown) of the stent 112. The fourth flexibility-enhancing row 126 includes a void 126a and a void 126b. The voids 126a and 126b may each extend only a short distance circumferentially around the stent 112. In some cases, the voids 126a and 126b may extend all the way around the stent 112 and join together on the back side (not shown) of the stent 112.

The stent 112 includes a total of two filaments extending in the first helical direction and two filaments extending in the second helical direction that cross each of the flexibility-enhancing circumferential rows of cells 120, 122, 124 and 126. This is similar to what is shown in FIG. 2. In some cases, there may be three such filaments extending in the first helical direction and crossing each circumferential row of cells that includes voids, much like what is shown in FIG. 6. Alternatively, in some cases there may be only a single filament extending in the first helical direction and crossing that circumferential row of cells, similar to what is shown in FIG. 4.

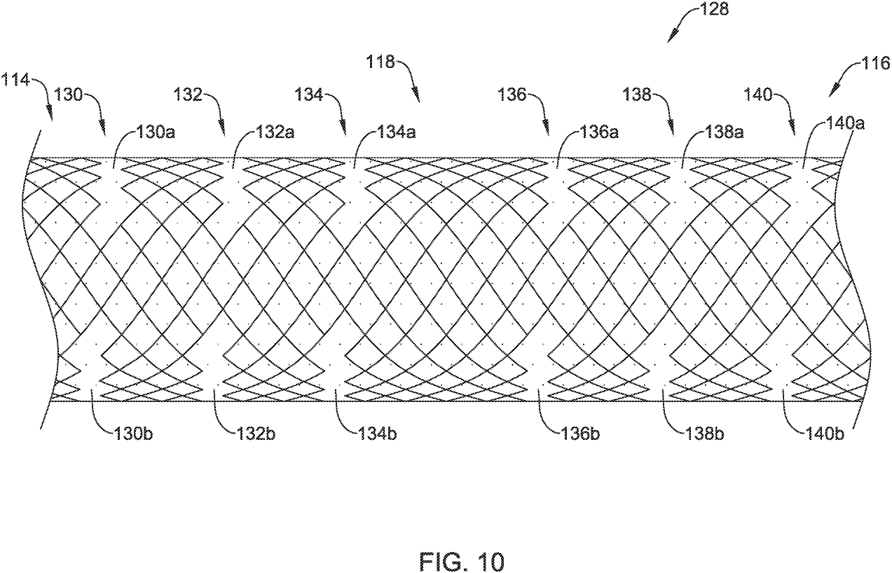

FIG. 10 is a side view of a portion of an illustrative stent 128 that includes a first or proximal region 114, a second or distal region 116 and an intervening intermediate region 118. The stent 128 may be expandable from a first radially collapsed configuration (not explicitly shown) to a second radially expanded configuration. In some cases, the stent 128 may be deployed to a configuration that is between the collapsed configuration and the expanded configuration, i.e., the stent 128 may be deployed having a deployed diameter that is greater than a diameter of the stent 128 or a particular portion thereof while in its collapsed configuration yet less than a diameter of the stent 128 or a particular portion thereof while in its fully expanded configuration. In some cases, the anatomy in which the stent 128 is deployed may influence its deployed configuration. For example, if the anatomy in which the stent 128 is to be deployed has a diameter that is less than a diameter of the stent 128 or a particular portion thereof when fully expanded, the stent 128 may have a deployed diameter that is intermediate its collapsed configuration diameter and its fully expanded configuration diameter.

In some cases, in the radially expanded configuration, the stent 128 may include anti-migration flared regions having enlarged diameters relative to a diameter of the illustrated portion of the stent 128. The anti-migration flared regions, if present, may be configured to engage an interior portion of the walls of the esophagus or other body lumen. The enlarged-diameter anti-migration regions can help to prevent the stent 128 from migrating once placed in the esophagus or other body lumen. In some instances, a transition to the enlarged diameters may be gradual, sloped, or occur in an abrupt step-wise manner, as desired.

In some instances, the first anti-migration flared region may have a first outer diameter and the second anti-migration flared region may have a second outer diameter. In some instances, the first and second outer diameters may be approximately the same, while in other instances, the first and second outer diameters may be different. In some cases, the stent 128 may include only one or none of the anti-migration flared regions. For example, a first end of the stent 128 may include an anti-migration flare while a second end of the stent 128 may not. In some cases, the second end of the stent 128 may include an anti-migration flare while the first end of the stent 128 does not. The stent 128 may have an outer diameter, outside of any flared regions, that is in the range of 15 to 25 millimeters in the fully expanded configuration. The outer diameter of any anti-migration flares may be in the range of 20 to 30 millimeters in the fully expanded configuration. It is contemplated that the outer diameter of the stent 128 may be varied to suit the desired application.

In order to enhance the flexibility of the stent 128, the stent 128 includes a plurality of flexibility-enhancing rows of cells in which at least some of the cells within each of the flexibility-enhancing rows have been disrupted. The stent 128 includes a first flexibility-enhancing row 130, a second flexibility-enhancing row 132, a third flexibility-enhancing row 134, a fourth flexibility-enhancing row 136, a fifth flexibility-enhancing row 138 and a sixth flexibility-enhancing row 140. While a total of six flexibility-enhancing rows 130, 132, 134, 136, 138 and 140 are illustrated, it will be appreciated that this is merely illustrative, as the stent 128 may have any desired number of flexibility-enhancing rows, including seven, eight, nine, ten or more flexibility-enhancing rows. In some instances, the stent 128 may have fewer than six flexibility-enhancing rows.

As seen, the first flexibility-enhancing row 130 includes a void 130a and a void 130b. The voids 130a and 130b may each extend only a short distance circumferentially around the stent 128. In some cases, the voids 130a and 130b may extend all the way around the stent 128 and join together on the back side (not shown) of the stent 128. The second flexibility-enhancing row 132 includes a void 132a and a void 132b. The voids 132a and 132b may each extend only a short distance circumferentially around the stent 128. In some cases, the voids 132a and 132b may extend all the way around the stent 128 and join together on the back side (not shown) of the stent 112. The third flexibility-enhancing row 134 includes a void 134a and a void 134b. The voids 134a and 134b may each extend only a short distance circumferentially around the stent 128. In some cases, the voids 134a and 134b may extend all the way around the stent 128 and join together on the back side (not shown) of the stent 128.

The fourth flexibility-enhancing row 136 includes a void 136a and a void 136b. The voids 136a and 136b may each extend only a short distance circumferentially around the stent 128. In some cases, the voids 136a and 136b may extend all the way around the stent 112 and join together on the back side (not shown) of the stent 112. The fifth flexibility-enhancing row 138 includes a void 138a and a void 138b. The voids 138a and 138b may each extend only a short distance circumferentially around the stent 128. In some cases, the voids 138a and 138b may extend all the way around the stent 112 and join together on the back side (not shown) of the stent 112. The sixth flexibility-enhancing row 140 includes a void 140a and a void 140b. The voids 140a and 140b may each extend only a short distance circumferentially around the stent 128. In some cases, the voids 140a and 140b may extend all the way around the stent 112 and join together on the back side (not shown) of the stent 112.

The stent 128 includes a total of two filaments extending in the first helical direction and two filaments extending in the second helical direction that cross each of the flexibility-enhancing rows 130, 132, 134, 136, 138 and 140. This is similar to what is shown in FIG. 2. In some cases, there may be three such filaments extending in the first helical direction and crossing each circumferential row of cells that includes voids, much like what is shown in FIG. 6. Alternatively, in some cases there may be only a single filament extending in the first helical direction and crossing that circumferential row of cells, similar to what is shown in FIG. 4.

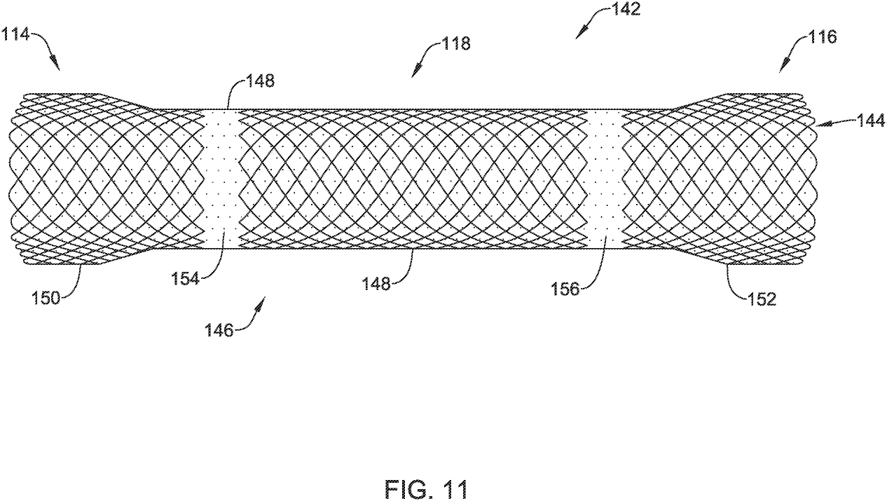

FIG. 11 is a side view of an illustrative endoluminal implant 142, such as but not limited to, a stent. The stent 142 may take the form of an elongated tubular member, although the stent 142 may take any cross-sectional shape desired. For example, the stent 142 may have a braided structure, fabricated from a plurality of filaments including a first plurality of filaments that each extend in a first helical direction and a second plurality of filaments that each extend in a second helical direction.

The stent 142 may have a first, or proximal end 114, a second, or distal end 116 and an intermediate region 118 that is disposed between the first end 114 and the second end 116. The stent 142 may include a lumen 144 that extends form a first opening adjacent the first end 114 to a second opening adjacent the second end 116 to allow for the passage of food, fluids, etc. to pass therethrough.

The stent 142 may be expandable from a first radially collapsed configuration (not explicitly shown) to a second radially expanded configuration. In some cases, the stent 142 may be deployed to a configuration that is between the collapsed configuration and the expanded configuration, i.e., the stent 142 may be deployed having a deployed diameter that is greater than a diameter of the stent 142 or a particular portion thereof while in its collapsed configuration yet less than a diameter of the stent 142 or a particular portion thereof while in its fully expanded configuration. In some cases, the anatomy in which the stent 142 is deployed may influence its deployed configuration. For example, if the anatomy in which the stent 142 is to be deployed has a diameter that is less than a diameter of the stent 142 or a particular portion thereof when fully expanded, the stent 142 may have a deployed diameter that is intermediate its collapsed configuration diameter and its fully expanded configuration diameter.

As shown in the radially expanded configuration, the stent 142 may include anti-migration flared regions having enlarged diameters relative to a diameter of the illustrated portion of the stent 142. The anti-migration flared regions, if present, may be configured to engage an interior portion of the walls of the esophagus or other body lumen. The enlarged-diameter anti-migration regions can help to prevent the stent 142 from migrating once placed in the esophagus or other body lumen. In some instances, a transition to the enlarged diameters may be gradual, sloped, or occur in an abrupt step-wise manner, as desired.

In some instances, the first anti-migration flared region may have a first outer diameter and the second anti-migration flared region may have a second outer diameter. In some instances, the first and second outer diameters may be approximately the same, while in other instances, the first and second outer diameters may be different. In some cases, the stent 142 may include only one or none of the anti-migration flared regions. For example, a first end of the stent 142 may include an anti-migration flare while a second end of the stent 142 may not. In some cases, the second end of the stent 142 may include an anti-migration flare while the first end of the stent 142 does not. The stent 142 may have an outer diameter, outside of any flared regions, that is in the range of 15 to 25 millimeters in the fully expanded configuration. The outer diameter of any anti-migration flares may be in the range of 20 to 30 millimeters in the fully expanded configuration. It is contemplated that the outer diameter of the stent 142 may be varied to suit the desired application.

In some cases, as illustrated, the stent 142 may be considered as including an elongate tubular member 146 and a polymeric covering 148 (shown via a dotted pattern) that covers the elongate tubular member 146. The polymeric covering 148 may be applied to the elongate tubular member 146 via dip coating or spray coating, for example. The elongate tubular member 146 includes a constant diameter segment 148, a first flared segment 150 and a second flared segment 152. The constant diameter segment 148 is distinct from the first flared segment 150 and the second flared segment 152, with a void space 154 disposed between the first flared segment 150 and the constant diameter segment 148 and a void space 156 disposed between the constant diameter segment 148 and the second flared segment 152.

In some cases, the constant diameter segment 148, the first flared segment 150 and the second flared segment 152 may each be independently braided with a first plurality of filaments extending in the first helical direction and a second plurality of filaments extending in the second helical direction. In some cases, the elongate tubular member 146 may be braided as a unitary member, with the same filaments extending through each of the first flared segment 150, the constant diameter segment 148 and the second flared segment 152, prior to cutting the elongate tubular member 146 into the distinct constant diameter segment 148, the first flared segment 150 and the second flared segment 152. It will be appreciated that the stent 142 will possess improved flexibility, due to the first void space 154 and the second void space 156 that allow the constant diameter segment 148, the first flared segment 150 and the second flared segment 152 to flex independently of each other. The polymeric covering 148 extends across each of the first void space 154 and the second void space 156.

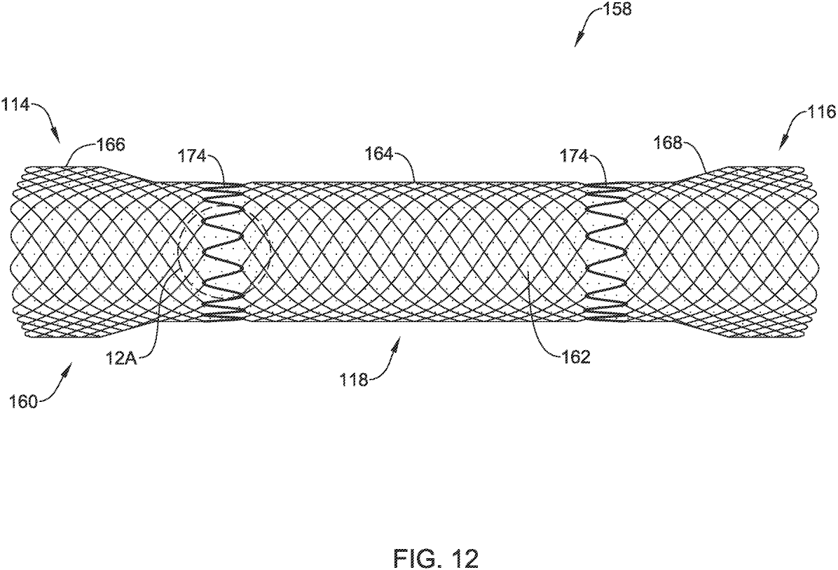

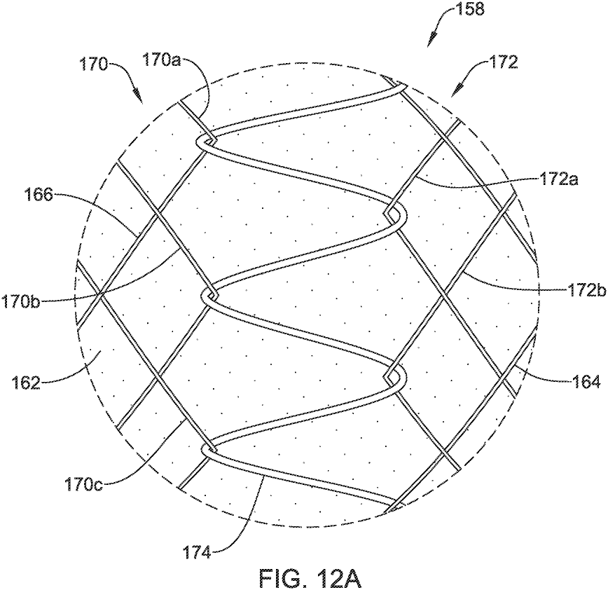

FIG. 12 is a side view of an illustrative endoluminal implant 158, such as but not limited to, a stent. The stent 158 may take the form of an elongated tubular member, although the stent 158 may take any cross-sectional shape desired. For example, the stent 158 may have a braided structure, fabricated from a plurality of filaments including a first plurality of filaments that each extend in a first helical direction and a second plurality of filaments that each extend in a second helical direction.

The stent 158 may have a first, or proximal end 114, a second, or distal end 116 and an intermediate region 118 that is disposed between the first end 114 and the second end 116. The stent 158 may include a lumen 144 that extends form a first opening adjacent the first end 114 to a second opening adjacent the second end 116 to allow for the passage of food, fluids, etc. to pass therethrough.

The stent 158 may be expandable from a first radially collapsed configuration (not explicitly shown) to a second radially expanded configuration. In some cases, the stent 158 may be deployed to a configuration that is between the collapsed configuration and the expanded configuration, i.e., the stent 158 may be deployed having a deployed diameter that is greater than a diameter of the stent 158 or a particular portion thereof while in its collapsed configuration yet less than a diameter of the stent 158 or a particular portion thereof while in its fully expanded configuration. In some cases, the anatomy in which the stent 158 is deployed may influence its deployed configuration. For example, if the anatomy in which the stent 158 is to be deployed has a diameter that is less than a diameter of the stent 158 or a particular portion thereof when fully expanded, the stent 158 may have a deployed diameter that is intermediate its collapsed configuration diameter and its fully expanded configuration diameter.

As shown in the radially expanded configuration, the stent 158 may include anti-migration flared regions having enlarged diameters relative to a diameter of the illustrated portion of the stent 158. The anti-migration flared regions, if present, may be configured to engage an interior portion of the walls of the esophagus or other body lumen. The enlarged-diameter anti-migration regions can help to prevent the stent 158 from migrating once placed in the esophagus or other body lumen. In some instances, a transition to the enlarged diameters may be gradual, sloped, or occur in an abrupt step-wise manner, as desired.

In some instances, the first anti-migration flared region may have a first outer diameter and the second anti-migration flared region may have a second outer diameter. In some instances, the first and second outer diameters may be approximately the same, while in other instances, the first and second outer diameters may be different. In some cases, the stent 158 may include only one or none of the anti-migration flared regions. For example, a first end of the stent 158 may include an anti-migration flare while a second end of the stent 158 may not. In some cases, the second end of the stent 158 may include an anti-migration flare while the first end of the stent 158 does not. The stent 158 may have an outer diameter, outside of any flared regions, that is in the range of 15 to 25 millimeters in the fully expanded configuration. The outer diameter of any anti-migration flares may be in the range of 20 to 30 millimeters in the fully expanded configuration. It is contemplated that the outer diameter of the stent 158 may be varied to suit the desired application.

In some cases, as illustrated, the stent 158 may be considered as including an elongate tubular member 160 and a polymeric covering 162 (illustrated via a dotted pattern) that covers the elongate tubular member 160. The polymeric covering 162 may be applied to the elongate tubular member 160 via dip coating or spray coating, for example. The elongate tubular member 160 includes a medial segment (e.g., constant diameter segment 164), a first end segment (e.g., a first flared segment 166) and a second end segment (e.g., a second flared segment 168), with the medial segment positioned between and spaced apart from the first and second end segments. The medial segment may not overlap the first end segment and/or the second end segment, such that a circumferential gap extending entirely around the circumference of the stent 158 is provided therebetween. The constant diameter segment 164 is distinct from the first flared segment 166 and the second flared segment 168.

In some cases, the constant diameter segment 164, the first flared segment 166 and the second flared segment 168 may each be independently braided with a first plurality of filaments extending in the first helical direction and a second plurality of filaments extending in the second helical direction. In some cases, the elongate tubular member 160 may be braided as a unitary member, with the same filaments extending through each of the first flared segment 166, the constant diameter segment 164 and the second flared segment 168, prior to cutting the elongate tubular member 160 into the distinct constant diameter segment 164, the first flared segment 166 and the second flared segment 168. It will be appreciated that the stent 158 will possess improved flexibility, due to the constant diameter segment 164, the first flared segment 166 and the second flared segment 168 being able to move independently of each other.

FIG. 12A shows an enlarged portion of the intersection between the first flared segment 166 and the constant diameter segment 164 of FIG. 12. The first flared segment 166 includes a terminal row 170 including a cell 170a, a cell 170b and a cell 170c. The constant diameter segment 164 includes a terminal row 172 including a cell 172a and a cell 172b. In some cases, as shown, the terminal row 172 is spaced somewhat apart from the terminal row 170, providing a circumferential gap extending entirely around the stent 158 between the first flared segment 166 and the constant diameter segment 164, and likewise, the terminal row of the second flared segment 168 is spaced apart from the terminal row of the constant diameter segment 164 providing a circumferential gap extending entirely around the stent 158 between the second flared segment 168 and the constant diameter segment 164. The cells 172a, 172b within the terminal row 172 are circumferentially offset from the cells 170a, 170b, 170c within the terminal row 170, which would allow the cells 172a, 172b to nest between the cells 170a, 170b, 170c if the terminal row 170 and the terminal row 172 were closer together. For instance, the peaks of the terminal row 170 may be longitudinally aligned with the valleys of the terminal row 172, and thus the valleys of the terminal row 170 may be longitudinally aligned with the peaks of the terminal row 172. Thus, each peak of the terminal row 170 may be circumferentially positioned between each peak of the terminal row 172 and each valley of the terminal row 170 may be circumferentially positioned between each valley of the terminal row 172. A similar arrangement may be present between the second flared segment 168 and the constant diameter segment 164.

In some cases, the stent 158 includes a fixation element 174 that joins together the terminal row 170 and the terminal row 172. As can be seen, the fixation element 174 may extend circumferentially around the circumference of the stent 158 while the fixation element 174 weaves in and out of the cells within the terminal row 170 and the terminal row 172 in order to secure the first flared segment 166 to the constant diameter segment 164. The fixation element 174 may zigzag back and forth across the circumferential gap as the fixation element crosses back and forth between the first flared segment 166 and the constant diameter segment 164. While not shown in an enlarged fashion, the constant diameter segment 164 may be secured to the second flared segment 168 in a similar manner. In some cases, the fixation element 174 may be a filament (e.g., a wire, a thread, or a suture) that can be used to essentially stitch the stent segments together. It will be appreciated that the polymeric covering 162 may also help to hold the stent segments together.

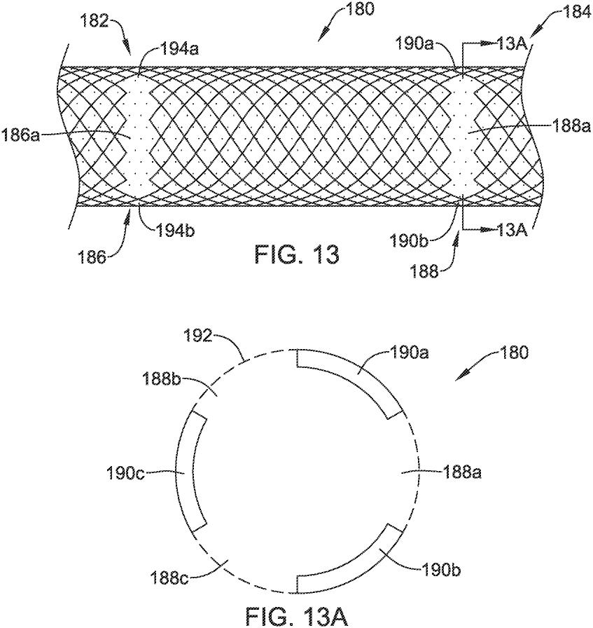

FIG. 13 is a side view of a portion of an illustrative stent 180 and FIG. 13A is a schematic cross-section thereof taken along the line 13A-13A of FIG. 13. The stent 180 may be expandable from a first radially collapsed configuration (not explicitly shown) to a second radially expanded configuration. The stent 180 may be considered as including a first region 182 and a second region 184. In some cases, the stent 180 may be deployed to a configuration that is between the collapsed configuration and the expanded configuration, i.e., the stent 180 may be deployed having a deployed diameter that is greater than a diameter of the stent 180 or a particular portion thereof while in its collapsed configuration yet less than a diameter of the stent 180 or a particular portion thereof while in its fully expanded configuration. In some cases, the anatomy in which the stent 180 is deployed may influence its deployed configuration. For example, if the anatomy in which the stent 180 is to be deployed has a diameter that is less than a diameter of the stent 180 or a particular portion thereof when fully expanded, the stent 180 may have a deployed diameter that is intermediate its collapsed configuration diameter and its fully expanded configuration diameter.

In some cases, in the radially expanded configuration, the stent 180 may include anti-migration flared regions having enlarged diameters relative to a diameter of the illustrated portion of the stent 180. The anti-migration flared regions, if present, may be configured to engage an interior portion of the walls of the esophagus or other body lumen. The enlarged-diameter anti-migration regions can help to prevent the stent 180 from migrating once placed in the esophagus or other body lumen. In some instances, a transition to the enlarged diameters may be gradual, sloped, or occur in an abrupt step-wise manner, as desired.

In some instances, the first anti-migration flared region may have a first outer diameter and the second anti-migration flared region may have a second outer diameter. In some instances, the first and second outer diameters may be approximately the same, while in other instances, the first and second outer diameters may be different. In some cases, the stent 180 may include only one or none of the anti-migration flared regions. For example, a first end of the stent 180 may include an anti-migration flare while a second end of the stent 180 may not. In some cases, the second end of the stent 180 may include an anti-migration flare while the first end of the stent 180 does not. The stent 180 may have an outer diameter, outside of any flared regions, that is in the range of 15 to 25 millimeters in the fully expanded configuration. The outer diameter of any anti-migration flares may be in the range of 20 to 30 millimeters in the fully expanded configuration. It is contemplated that the outer diameter of the stent 180 may be varied to suit the desired application.

In order to enhance the flexibility of the stent 180, the stent 180 includes a first flexibility-enhancing row 186 that is located within the first region 182 of the stent 180 and a second flexibility-enhancing row 188 that is located within the second region 184 of the stent 180. The stent 180 may include additional flexibility-enhancing rows as well. A polymeric covering 192 extends circumferentially around the stent 180. As can be seen in FIG. 13A, the second flexibility-enhancing row 188 includes a total of three voids, individually labeled as 188a, 188b and 188c that are disposed between stent wall segments 190a, 190b and 190c. While shown with each of the voids 188a, 188b and 188c each having an arc length that is about the same as an arc length of each of the stent wall segments 190a, 190b and 190c, this is just an example. In some cases, the voids 188a, 188b and 188c may be relatively longer and the stent wall segments 190a, 190b and 190c may be relatively shorter. In some cases, the voids 188a, 188b and 188c may be relatively shorter and the stent wall segments 190a, 190b and 190c may be relatively longer. It will be appreciated that the first flexibility-enhancing row 186 similarly includes a total of three voids, although only a single void 186a is visible in this orientation. The first flexibility-enhancing row 186 includes a total of three stent wall segments, although only two stent wall segments 194a and 194b are visible in this orientation.

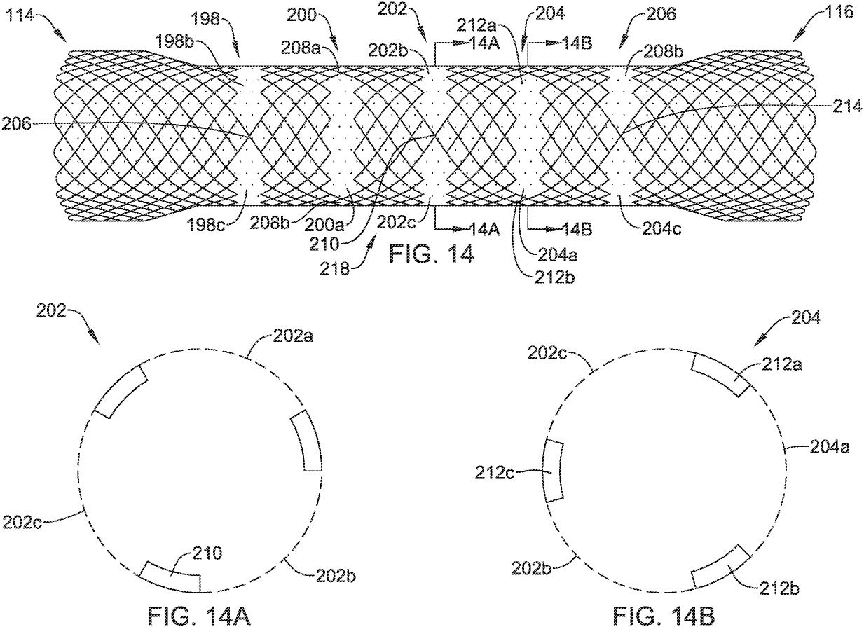

FIG. 14 is a side view of an illustrative stent 196, FIG. 14A is a schematic cross-section of the stent 196 taken along the line 14A-14A of FIG. 14, and FIG. 14B is a schematic cross-section of the stent 196 taken along the line 14B-14B of FIG. 14. The stent 196 may have a first, or proximal end 114, a second, or distal end 116 and an intermediate region 118 that is disposed between the first end 114 and the second end 116. The stent 196 may include a lumen 144 that extends form a first opening adjacent the first end 114 to a second opening adjacent the second end 116 to allow for the passage of food, fluids, etc. to pass therethrough.

The stent 196 may be expandable from a first radially collapsed configuration (not explicitly shown) to a second radially expanded configuration. In some cases, the stent 196 may be deployed to a configuration that is between the collapsed configuration and the expanded configuration, i.e., the stent 196 may be deployed having a deployed diameter that is greater than a diameter of the stent 196 or a particular portion thereof while in its collapsed configuration yet less than a diameter of the stent 196 or a particular portion thereof while in its fully expanded configuration. In some cases, the anatomy in which the stent 196 is deployed may influence its deployed configuration. For example, if the anatomy in which the stent 196 is to be deployed has a diameter that is less than a diameter of the stent 196 or a particular portion thereof when fully expanded, the stent 196 may have a deployed diameter that is intermediate its collapsed configuration diameter and its fully expanded configuration diameter.

As shown in the radially expanded configuration, the stent 196 may include anti-migration flared regions having enlarged diameters relative to a diameter of the illustrated portion of the stent 196. The anti-migration flared regions, if present, may be configured to engage an interior portion of the walls of the esophagus or other body lumen. The enlarged-diameter anti-migration regions can help to prevent the stent 196 from migrating once placed in the esophagus or other body lumen. In some instances, a transition to the enlarged diameters may be gradual, sloped, or occur in an abrupt step-wise manner, as desired.

In some instances, the first anti-migration flared region may have a first outer diameter and the second anti-migration flared region may have a second outer diameter. In some instances, the first and second outer diameters may be approximately the same, while in other instances, the first and second outer diameters may be different. In some cases, the stent 196 may include only one or none of the anti-migration flared regions. For example, a first end of the stent 196 may include an anti-migration flare while a second end of the stent 196 may not. In some cases, the second end of the stent 196 may include an anti-migration flare while the first end of the stent 196 does not. The stent 196 may have an outer diameter, outside of any flared regions, that is in the range of 15 to 25 millimeters in the fully expanded configuration. The outer diameter of any anti-migration flares may be in the range of 20 to 30 millimeters in the fully expanded configuration. It is contemplated that the outer diameter of the stent 196 may be varied to suit the desired application.

In order to enhance the flexibility of the stent 196, the stent 196 includes a first flexibility-enhancing row 198, a second flexibility-enhancing row 200, a third flexibility-enhancing row 202, a fourth flexibility-enhancing row 204 and a fifth flexibility-enhancing row 206. The stent 196 may include additional flexibility-enhancing rows. In some cases, the stent 196 may include fewer flexibility-enhancing rows. In some cases, as illustrated, each flexibility-enhancing row 198, 200, 202, 204, 206 is circumferentially rotated relative to an adjacent flexibility-enhancing row 198, 200, 202, 204, 206. Because FIG. 14A is a cross-sectional view taken through the flexibility-enhancing row 202 and FIG. 14B is a cross-sectional view taken through the flexibility-enhancing row 204, the relative rotation between adjacent flexibility-enhancing rows is easy to see.

In comparing FIG. 14A with FIG. 14B, it is easy to see that the flexibility-enhancing row 204 is rotated clockwise relative to the flexibility-enhancing row 202. Each of the flexibility-enhancing rows 198, 200, 202, 204, 206 include several voids and several intervening stent wall segments. The voids may be considered as being windows that are cut into the stent 196, such as via laser cutting or saw cutting. Rather than necessarily cutting individual filaments adjacent a crossing point between a particular filament and another filament, the windows formed within the stent 196 may be cut independently of where the crossing points are, and may for example be rectilinear in shape. Other shapes are also contemplated. A polymeric coating 216 that envelopes the stent 196 may be seen as extending through the voids or windows cut into the stent 196.

To illustrate, the first flexibility-enhancing row 198 has a pair of voids 198b and 198c visible (with a third void 198a positioned out of sight behind the stent 198, with a single stent wall segment 206 visible between the void 198b and the void 198c. The second flexibility-enhancing row 200 has a single void 200a visible between a stent wall segment 208a and a stent wall segment 208b.

The third flexibility-enhancing row 202 has a pair of voids 202b and 202c visible (with a third void 202a positioned out of sight behind the stent 198, with a single stent wall segment 210 visible between the void 202b and the void 202c. The fourth flexibility-enhancing row 204 has a single void 240a visible between a stent wall segment 212a and a stent wall segment 212b. The fifth flexibility-enhancing row 206 has a pair of voids 206b and 202c visible (with a third void 206a positioned out of sight behind the stent 198, with a single stent wall segment 214 visible between the void 206b and the void 206c.

As shown, each of the stent wall segments, including the stent wall segments 212a, 212b, 212c, 206, 210 and 214 have an arc length that is less than an arc length of the voids dispersed between each of the stent wall segments 212a, 212b, 212c, 206, 210 and 214. In some cases, each of the stent wall segments, including the stent wall segments 212a, 212b, 212c, 206, 210 and 214 may have an arc length that is about equal to an arc length of the voids dispersed between each of the stent wall segments 212a, 212b, 212c, 206, 210 and 214. In some cases, each of the stent wall segments, including the stent wall segments 212a, 212b, 212c, 206, 210 and 214 have an arc length that is greater than an arc length of the voids dispersed between each of the stent wall segments 212a, 212b, 212c, 206, 210 and 214.

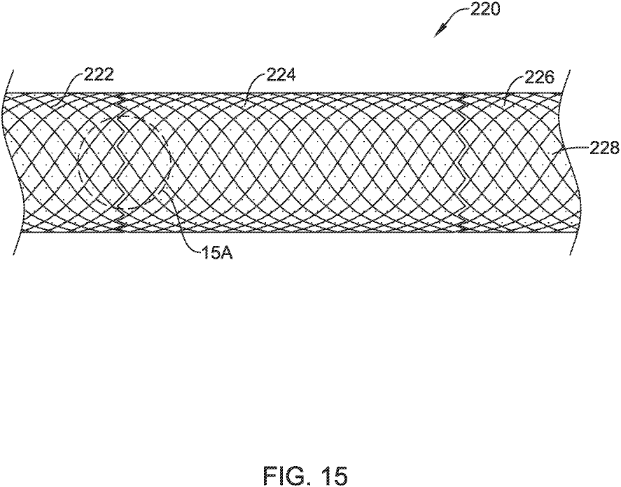

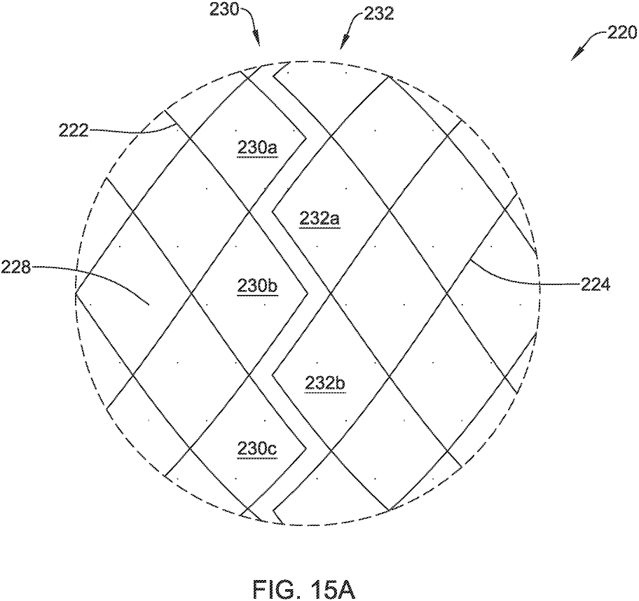

FIG. 15 is a side view of a portion of an illustrative stent 220. The stent 220 includes a first segment 222, a second segment 224 and a third segment 226. Each of the first segment 222, the second segment 224 and the third segment 226 may be formed by braiding together a first plurality of filaments extending in the first helical direction and a second plurality of filaments extending in the second helical direction prior to cutting free each of the first segment 222, the second segment 224 and the third segment 226. In some cases, each of the first segment 222, the second segment 224 and the third segment 226 may be separately formed by braiding together a first plurality of filaments extending in the first helical direction and a second plurality of filaments extending in the second helical direction to form the first segment 222, and braiding together a first plurality of filaments extending in the first helical direction and a second plurality of filaments extending in the second helical direction to form the second segment 224, and also braiding together a first plurality of filaments extending in the first helical direction and a second plurality of filaments extending in the second helical direction to form the third segment 226. In either event, a polymeric covering 228 envelops the stent 220.

FIG. 15A is an enlarged view of a portion of the stent 220, showing the relationship between the first segment 222 and the second segment 224. The first segment 222 includes a terminal row 230 that includes (as visible) a cell 230a, a cell 230b and a cell 230c. The second segment 224 includes a terminal row 232 having (as shown) a cell 232a and a cell 232b. It will be appreciated that the terminal row 230 and the terminal row 232, as well as the cells forming those terminal rows, continue circumferentially around the stent 220. It will be appreciated that the cells 232a, 232b within the terminal row 232 are circumferentially offset from the cells 230a, 230b, 230c within the terminal row 230. This allows the cells 232a, 232b to nest between the cells 230a, 230b, 230c. Thus, the peaks of the terminal row 230 of the first segment 222 may be located closer to the third segment 226 than the peaks of the terminal row 232 of the second segment 224. For instance, the peaks of the terminal row 230 may be longitudinally aligned with the valleys of the terminal row 232, and thus the valleys of the terminal row 230 may be longitudinally aligned with the peaks of the terminal row 232. Thus, each peak of the terminal row 230 may be circumferentially positioned between each peak of the terminal row 232 and each valley of the terminal row 230 may be circumferentially positioned between each valley of the terminal row 232. A similar configuration and arrangement may be provided between the second segment 224 and the third segment 226.

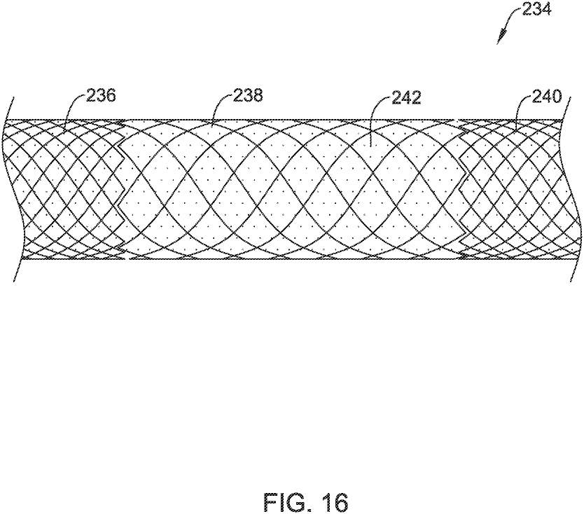

FIG. 16 is a side view of a portion of an illustrative stent 234. The stent 234 includes a first segment 236, a second segment 238 and a third segment 240 that are coupled together via a polymeric covering 242 (shown via a dotted pattern). Unlike FIG. 15, in which the segments 222, 224 and 226 shared a braiding pattern, the segments 236, 238 and 240 of FIG. 16 may not share a braiding pattern. As shown, the first segment 236 and the third segment 240 have a similar if not identical braiding pattern while the second segment 238 has a different braiding pattern in which fewer filaments are braided together, forming a second segment 238 having larger cells between adjacent filaments. In this particular case, the first segment 236 and the third segment 240 could be formed by braiding together a first plurality of filaments extending in the first helical direction and a second plurality of filaments extending in the second helical direction prior to cutting free each of the first segment 236 and the third segment 240. The second segment 238 would be separately braided. In some cases, each of the first segment 236, the second segment 238 and the third segment 240 may be separately braided before being assembled together.

In some instances, the terminal row of the second segment 238 adjacent the first segment 236 may include cells nested between cells in the terminal row of the first segment 236. For instance, the peaks of the terminal row of the second segment 238 adjacent the first segment 236 may be longitudinally aligned with valleys of the terminal row of the first segment 236. As illustrated, the terminal row of the second segment 238 may include fewer peaks than the terminal row of the first segment 236 such that not every valley of the terminal row of the first segment 236 receives a peak of the terminal row of the second segment 238. Thus, each peak of the terminal row of the second segment 238 may be circumferentially positioned between adjacent peaks of the terminal row of the first segment 236. Other configurations are also contemplated.

In some instances, the terminal row of the second segment 238 adjacent the third segment 240 may include cells nested between cells in the terminal row of the third segment 240. For instance, the peaks of the terminal row of the second segment 238 adjacent the third segment 240 may be longitudinally aligned with valleys of the terminal row of the third segment 240. As illustrated, the terminal row of the second segment 238 may include fewer peaks than the terminal row of the third segment 240 such that not every valley of the terminal row of the third segment 240 receives a peak of the terminal row of the second segment 238. Thus, each peak of the terminal row of the second segment 238 may be circumferentially positioned between adjacent peaks of the terminal row of the third segment 240. Other configurations are also contemplated.

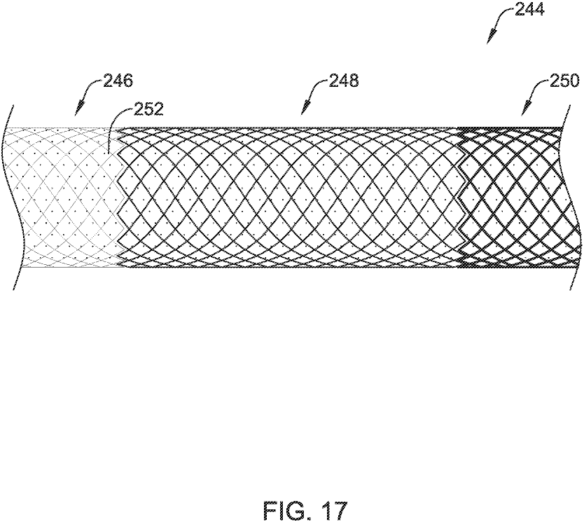

FIG. 17 is a side view of a portion of an illustrative stent 244. The stent 244 includes a first segment 246, a second segment 248 and a third segment 250 that are coupled together via a polymeric covering 252 (shown via a dotted pattern). In this particular example, each of the first segment 246, the second segment 248 and the third segment 250 share a similar braiding pattern, but with different filaments. The filaments used to form the first segment 246 are a lighter weight than the filaments that are used to form the second segment 248. Similarly, the filaments used to form the second segment 248 are a lighter weight than the filaments that are used to form the third segment 250. As a result, it will be appreciated that each segment will have different properties. In some cases, braiding patterns may vary by varying one or more of wire count, wire diameter, braid angle and others.

In some instances, the terminal row of the second segment 248 adjacent the first segment 246 may include cells nested between cells in the terminal row of the first segment 246. For instance, the peaks of the terminal row of the second segment 248 adjacent the first segment 246 may be longitudinally aligned with valleys of the terminal row of the first segment 246. Thus, each peak of the terminal row of the second segment 248 may be circumferentially positioned between adjacent peaks of the terminal row of the first segment 246. Other configurations are also contemplated.

In some instances, the terminal row of the second segment 248 adjacent the third segment 250 may include cells nested between cells in the terminal row of the third segment 250. For instance, the peaks of the terminal row of the second segment 248 adjacent the third segment 250 may be longitudinally aligned with valleys of the terminal row of the third segment 250. Thus, each peak of the terminal row of the second segment 248 may be circumferentially positioned between adjacent peaks of the terminal row of the third segment 250. Other configurations are also contemplated.

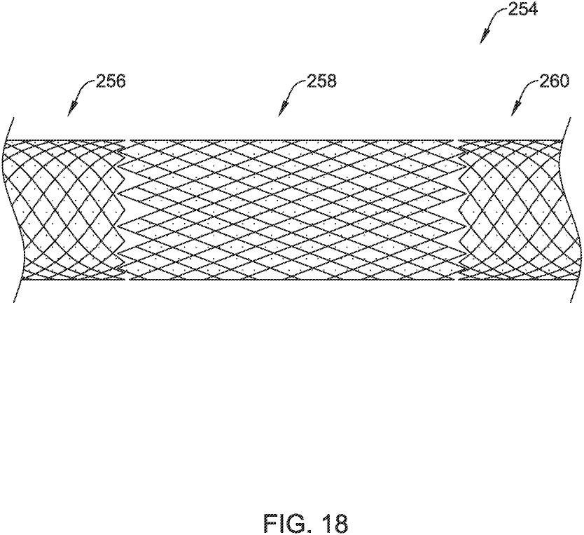

FIG. 18 is a side view of a portion of an illustrative stent 254. The stent 254 includes a first segment 256, a second segment 258 and a third segment 260 that are coupled together via a polymeric covering 262 (shown via a dotted pattern). In this particular example, the first segment 256 and the third segment 260 share a similar braiding pattern, but the second segment 258 has a different braiding pattern in which a greater number of filaments are braided together. It will be appreciated that the second segment 258 will have properties that are different from those of the first segment 256 and the third segment 260.

In some instances, the terminal row of the second segment 258 adjacent the first segment 256 may include cells nested between cells in the terminal row of the first segment 256. For instance, the peaks of the terminal row of the second segment 258 adjacent the first segment 256 may be longitudinally aligned with valleys of the terminal row of the first segment 256. Thus, each peak of the terminal row of the second segment 258 may be circumferentially positioned between adjacent peaks of the terminal row of the first segment 256. Other configurations are also contemplated.

In some instances, the terminal row of the second segment 258 adjacent the third segment 260 may include cells nested between cells in the terminal row of the third segment 260. For instance, the peaks of the terminal row of the second segment 258 adjacent the third segment 260 may be longitudinally aligned with valleys of the terminal row of the third segment 260. Thus, each peak of the terminal row of the second segment 258 may be circumferentially positioned between adjacent peaks of the terminal row of the third segment 260. Other configurations are also contemplated.

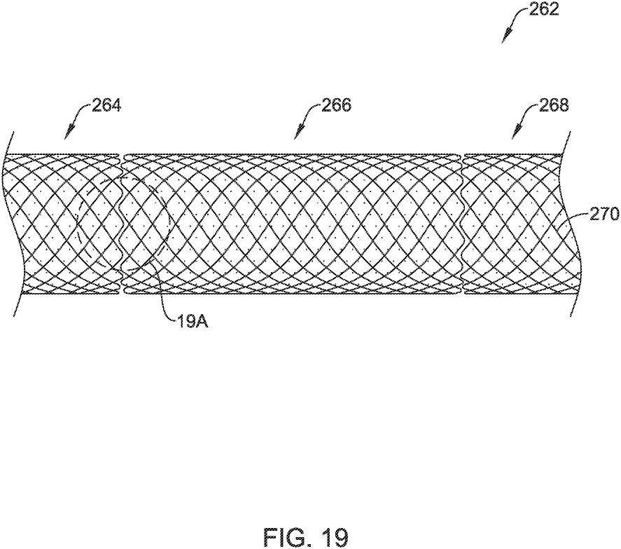

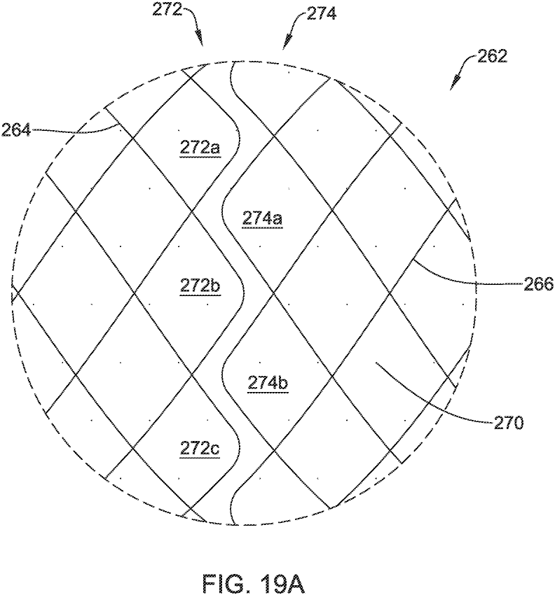

FIG. 19 is a side view of a portion of an illustrative stent 262. The stent 262 includes a first segment 264, a second segment 266 and a third segment 268 that are coupled together via a polymeric covering 270 (shown via a dotted pattern). In this particular example, the three segments 264, 266 and 268 share a similar braiding pattern, but the relationships between the three segments 264, 266 and 268 are a little different than those shown in FIGS. 15-18. Rather than having a rather abrupt transition between adjoining segments, in FIG. 19 the braiding patterns have been modified somewhat to provide a more atraumatic end to each of the segments 264, 266, 268.

FIG. 19A is an enlarged view of a portion of the stent 262 showing the relationship between the first segment 264 and the second segment 266. The first segment 264 includes a terminal row 272 that includes (as shown) a cell 272a, a cell 272b and a cell 272c. The second segment 264 includes a terminal row 274 that includes (as shown) a cell 274a and a cell 274b. It will be appreciated that the terminal row 272 and the terminal row 274, as well as the cells forming those terminal rows, continue circumferentially around the stent 262. It will be appreciated that the cells 274a and 274b within the terminal row 274 are circumferentially offset from the cells 272a, 272b and 272c within the terminal row 272. This allows the cells 274a, 274b to nest between the cells 272a, 272b, 272c. For instance, the peaks of the terminal row 272 may be longitudinally aligned with the valleys of the terminal row 274, and thus the valleys of the terminal row 272 may be longitudinally aligned with the peaks of the terminal row 274. Thus, each peak of the terminal row 272 may be circumferentially positioned between each peak of the terminal row 274 and each valley of the terminal row 272 may be circumferentially positioned between each valley of the terminal row 274. Moreover, each of the cells 272a, 272b, 272c within the terminal row 272 and the cells 274a and 274b within the terminal row 272 have been modified to have more gently curved, atraumatic, edges.

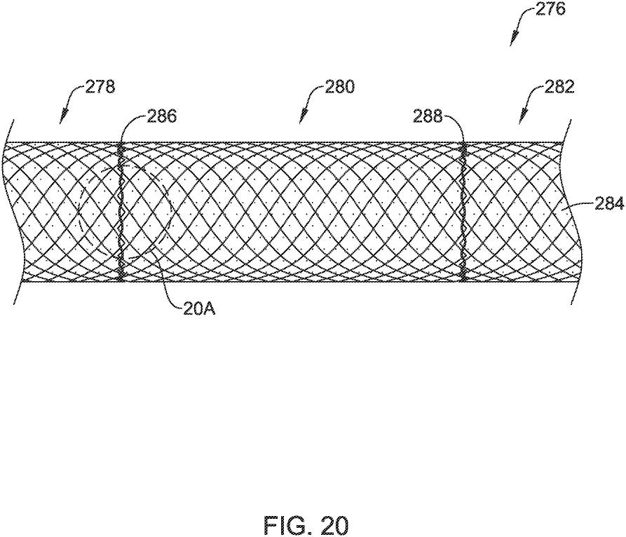

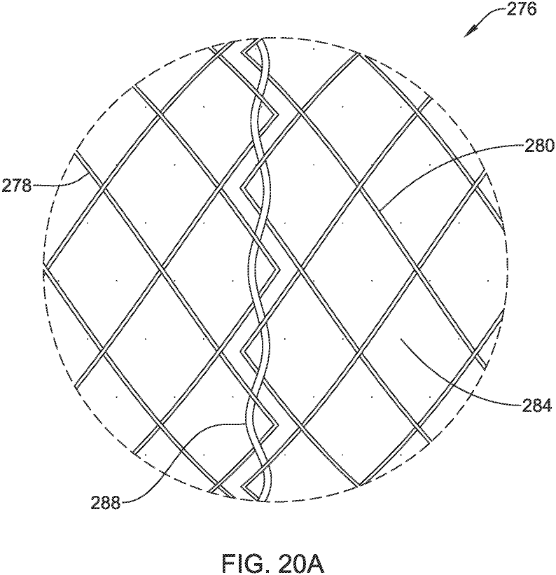

FIG. 20 is a side view of a portion of an illustrative stent 276 and FIG. 20A is an enlarged view of a portion thereof. The stent 276 includes a first segment 278, a second segment 280 and a third segment 282 that are coupled together at least in part via a polymeric covering 284 (shown via a dotted pattern). In some ways, the stent 276 is similar to the stent 220 shown in FIG. 15. However, the stent 276 includes fixation elements 286 and 288 extending circumferentially around the circumference of the stent 276 that weave back and forth adjoining the first segment 278 to the second segment 280 and adjoining the second segment 280 to the third segment 282, respectively. In some cases, the fixation elements 286 and 288 may be filaments (e.g., wires, threads or sutures), for example. As can be seen for example in FIG. 20A, the fixation element 288 (and the fixation element 286) extends circumferentially around the circumference of the stent 276 while the fixation element 288 (and the fixation element 286) weaves back and forth through the cells to adjoin the adjacent segments.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention’s scope is, of course, defined in the language in which the appended claims are expressed.

What is claimed is:

1. A stent, comprising:
an elongated tubular member expandable from a radially collapsed configuration to a radially expanded configuration, the elongate tubular member comprising a first plurality of filaments extending in a first helical direction and a second plurality of filaments extending in a second helical direction, the first plurality of filaments extending in the first helical direction and the second plurality of filaments extending in the second helical direction overlapping to form a plurality of cells arranged in rows extending circumferentially about the elongated tubular member;
wherein at least some of the cells within one or more rows are adapted to provide increased flexibility to the stent.

2. The stent of claim 1, wherein at least some of the first plurality of filaments and at least some of the second plurality of filaments within one or more rows of the plurality of rows are cut away to disrupt at least some of the cells, thereby increasing flexibility of the stent.

3. The stent of claim 1, wherein at least some of the plurality of cells have a generally diamond shape having four sides formed by a pair of filaments of the first plurality of filaments extending in the first helical direction and a pair of filaments of the second plurality of filaments extending in the second helical direction.

4. The stent of claim 3, wherein a disrupted cell comprises a cell that has had at least one of the four sides of the generally diamond shape removed.

5. The stent of claim 1, wherein the stent further comprises a polymeric covering extending along the elongate tubular member.

6. The stent of claim 1, wherein one or more rows of cells include at least one intact cell and a plurality of disrupted cells.

7. The stent of claim 1, wherein one or more rows of cells include only disrupted cells, thereby separating the elongate tubular member into two or more distinct segments.

8. The stent of claim 7, wherein:
a first segment of the two or more distinct segments has a first end having a plurality of cells within a first terminal row;
a second segment of the two or more distinct segments has a second end having a plurality of cells within a second terminal row; and
wherein the second segment is rotated relative to the first segment such that the plurality of cells within the first terminal row nest between the plurality of cells within the second terminal row.

9. The stent of claim 8, wherein a first segment of the two or more distinct segments have a first braiding pattern and a second segment of the two or more distinct segments have a second braiding pattern that is different from the first braiding pattern.

10. The stent of claim 9, wherein the first braiding pattern differs from the second braiding pattern in one or more of filament count, braid angle, and filament diameter.

11. The stent of claim 8, wherein the two or more distinct segments are joined together via a fixation element woven between adjacent segments.

12. The stent of claim 11, wherein the fixation element comprises a filament.

13. The stent of claim 8, wherein the two or more distinct segments are formed by disrupting all of the cells within a row of cells.

14. A braided stent, comprising
an elongated tubular member expandable from a radially collapsed configuration to a radially expanded configuration, the elongate tubular member including:
a first segment comprising a first plurality of filaments extending in a left to right helical direction and a second plurality of filaments extending in a right to left helical direction, the first plurality of filaments and the second plurality of filaments together forming a first plurality of cells arranged in rows extending circumferentially about the first segment; and
a second segment comprising a third plurality of filaments extending in the left to right helical direction and a fourth plurality of filaments extending in the right to left helical direction, the third plurality of filaments and the fourth plurality of filaments together forming a second plurality of cells arranged in rows extending circumferentially about the second segment;
wherein the first segment and the second segment are coupled together in a manner that provides the braided stent with increased flexibility.

15. The braided stent of claim 14, wherein the first segment and the second segment are coupled together by having one or more of the third plurality of filaments being extensions of one or more of the first plurality of filaments and/or by having one or more of the fourth plurality of filaments being extensions of one or more of the second plurality of filaments.

16. The braided stent of claim 14, wherein the first segment and the second segment are coupled together by a continuous polymeric layer that extends over at least part of the first segment and at least part of the second segment.

17. The braided stent of claim 14, wherein:
the first segment has a first end having a plurality of cells within a first terminal row;
the second segment has a second end having a plurality of cells within a second terminal row; and
the first segment and the second segment are coupled together via a fixation element woven between the plurality of cells within the first terminal row and the plurality of cells within the second terminal row.

18. The braided stent of claim 14, further comprising a third segment comprising a fifth plurality of filaments extending in a left to right helical direction and a sixth plurality of filaments extending in a right to left helical direction, the first plurality of filaments and the second plurality of filaments together forming a first plurality of cells arranged in rows extending circumferentially about the first segment;
wherein the second segment and the third segment are coupled are coupled together in a manner that provides the braided stent with increased flexibility.

19. A braided stent, comprising:
an elongated tubular member expandable from a radially collapsed configuration to a radially expanded configuration, the elongate tubular member comprising a plurality of cells arranged in rows that extend circumferentially about the elongated tubular member;
wherein at least some of the plurality of cells are adapted to increase flexibility of the stent.

20. The braided stent of claim 19, wherein the elongated tubular member comprises a first plurality of filaments extending in a first helical direction and a second plurality of filaments extending in a second helical direction, the first plurality of filaments extending in the first helical direction and the second plurality of filaments extending in the second helical direction overlapping to form a plurality of cells arranged in rows extending circumferentially about the elongated tubular member;
wherein at least some of the first plurality of filaments and at least some of the second plurality of filaments within one or more rows of the plurality of rows are cut away to disrupt at least some of the cells, thereby increasing flexibility of the stent.
